(19) United States
(12) Patent Application Publication
(10) Pub. No.: US 2008/0261319 A1
(43) Pub. Date: Oct. 23, 2008

(54) METHOD FOR DETECTING PRESENCE OF ARISTOLOCHIA MATERIALS IN HERBAL PRODUCTS AND BOTANICALS

(76) Inventors: Paul Pui-Hay But, Hong Kong SAR (HK); Pang Chiu Shaw, Hong Kong SAR (HK); Ming Li, Hong Kong SAR (HK); Hilary Lam, Hong Kong SAR (HK); Joyce Wing-Hin But, Hong Kong SAR (HK)

Correspondence Address: EVAN LAW GROUP LLC, 600 WEST JACKSON BLVD., SUITE 625, CHICAGO, IL, 60661, US

(21) Appl. No.: 11/738,754

(22) Filed: Apr. 23, 2007

(51) Int. Cl. G01N 33/00; C07H 21/02

(52) U.S. Cl. 436/94; 536/23.1

(57) ABSTRACT

A method for detecting herbs or plants of the Aristolochia genus in herbal products and botanicals may be based on the sequence information in the trnL-trnF region and/or the psbA-trnH region. The trnL-trnF region and/or the psbA-trnH of the chloroplast DNA contain a number of segments which are highly characteristic of the plants of the Aristolochia genus. By this method, the degree of similarity in one or more of these segments between an herbal or plant material of unknown identification and reference DNA sequence of a known material can be used to authenticate the unknown material.

### TECHNICAL FIELD

This invention relates to a method for distinguishing herbal products and botanicals. Particularly, it relates to a molecular method for distinguishing plant materials of the Aristolochia genus from herbs derived from other plant families used in Chinese medicine. The method is based on the sequences of the trnL-trnF region and the psbA-trnH region of the chloroplast DNA.

### BACKGROUND

Herbal materials from the Aristolochia genus are known to contain aristolochic acids and related chemical compounds that could induce nephropathy and carcinoma. In Traditional Chinese Medicine (TCM), materials derived from Aristolochia species are sometimes used as alternatives or adulterants of other herbal products and botanicals such as Muxiang, Mutong, Fangji, Baiying, Madouling and Zhushalian. The relevant information is as follows:
1) The herb Muxiang can come from the roots of Aucklandia lappa (Muxiang) and other species of the daisy family (Compositae), and Aristolochia debilis (Qingmuxiang) and related Aristolochia species.
2) The herb Mutong can come from the roots of Akebia quinata, Akebia trifoliata, Akebia trifoliata var. australis (Mutong), Clematis armandii Clematis montana (Chuanmutong), and Aristolochia manshuriensis (Guangmutong) and related Aristolochia species.
3) The herb Fangji can come from the roots of Stephania tetrandra (Fangji) and Aristolochia fangchi (Guangfangji) and related Aristolochia species.
4) The herb Baiying should be from Solanum lyratum but it has sometimes been confused with another herb Xungufeng derived from Aristolochia mollissima and related Aristolochia species.
5) The herb Madouling can come from the fruits of Aristolochia contorta, Aristolochia debilis, and Dabaihe derived from the fruits or seeds of Cardiocrinum and related taxa in Liliaceae.
6) The Herb Zhushalian can come from the root of Aristolochia kaempferi and related species of Aristolochia and may be confused by Dioscorea cirrhosa and related species in Dioscoreaceae.

Since the early 1990s, hundreds of poisoning cases due to consumption of herbs and botanicals derived from or containing Aristolochia plants were reported in many parts of the world. The toxic substances in the Aristolochia plants are aristolochic acids and related compounds. In many countries and regions, plant materials from Aristolochia genus are banned in all herbal products and botanicals. For example, they are banned in Hong Kong and Taiwan. In mainland China, however, some Aristolochia species are still allowed for use as herbal products and botanicals. Where such Aristolochia materials are permitted to be used as herb medicines, it should be advised that the true source identity of such materials be revealed so that particular caution can be exercised on the part of the doctor and the patient. For the countries where all Aristolochia materials are banned, there are chances for inadvertent shipment or mix of materials derived from Aristolochia species. It is thus important to be able to tell if a particular herbal product and botanical contains materials derived from plant species belonging to the genus of Aristolochia. Thus, an accurate and definitive method is needed to reveal their presence in herbal products and botanicals to help protect consumers, to reinforce good manufacturing practices (GMP), and to generate evidences for legal proceedings.

The existing methods in identifying Chinese herbal medicines are usually based on organoleptic approaches which are very much dependent on subjective judgment of the inspectors or dispensers. Morphological characters for identification are usually of limited value because the genuine herbal species and its adulterants are usually sharing very similar characters. Anatomical and chemical (e.g. TLC, HPLC, GC-MS) studies are affected by factors such as the growing stage and the post-harvest processing.

### SUMMARY

Accordingly, an object of the present invention is to provide a method that can determine whether an herbal material is derived from a plant of the Aristolochia genus or whether an herbal product and botanical contains a material derived from an Aristolochia plant.

This and other objects of the present invention is realized by making use of the sequence information in the trnL-trnF and the psbA-trnH regions of the chloroplast DNA to distinguish Aristolochia materials from other herbal materials commonly used in TCM.

According to an embodiment of the present invention, the method involves the following steps: (a) obtaining a set of reference (or standard) DNA sequences; (b) extracting total DNA from one or more herbal samples in question; (c) amplifying the trnL-trnF and/or the psbA-trnH regions with a polymerase chain reaction (PCR) technology or other technologies; (d) determining the nucleotide sequences of the amplified DNA regions; (e) aligning the nucleotide sequences of the trnL-trnF and psbA-trnH regions (or a portion of the regions) of the samples with the reference DNA sequences; and (f) optionally, generating a dendrogram based on the nucleotide sequences of the trnL-trnF and psbA-trnH regions of the samples and the references. In a typical example, after the DNA extraction, PCR amplification and DNA sequencing steps, one can compare and match the resulting trnL-trnF and/or psbA-trnH sequences of the sample to those of the reference DNA sequences of the Aristolochia herbs. If a match is found, the sample belongs to Aristolochia.

As used here, the term “reference DNA sequence” refers to a DNA sequence which is, or encompasses, or is part of, the trnL-trnF region or the psbA-trnH region of the chloroplast DNA of an authenticated herbal material of a plant species.

The methods and techniques suitable for amplifying the trnL-trnF and/or the psbA-trnH regions are well known and are within ordinary skill of people in the art, as are the methods and techniques for DNA sequence alignment and generating dendrograms using computer programs such as BioEdit and MEGA.

In sum, with the method of the present invention, any herbal medicines derived from an Aristolochia species can be detected and distinguished from herbal medicines derived from other plant species, as exemplified by the examples described in the following:

### BRIEF DESCRIPTION OF THE DRAWINGS

FIG. 1a shows a trnL-trnF sequence alignment of Muxiang and related Aristolochia species.

FIG. 1b shows a dendrogram of Muxiang and related Aristolochia species constructed based on the trnL-trnF sequence alignment.

FIG. 2a shows a psbA-trnH sequence alignment of Muxiang and related Aristolochia species.

FIG. 2b shows a dendrogram of Muxiang and related Aristolochia species constructed based on the psbA-trnH sequence alignment.

FIG. 3a shows a trnL-trnF sequence alignment of Mutong and related Aristolochia species.

FIG. 3b shows a dendrogram of Mutong and related Aristolochia species constructed based on the trnL-trnF sequence alignment.

FIG. 4a shows a psbA-trnH sequence alignment of Mutong and related Aristolochia species.

FIG. 4b shows a dendrogram of Mutong and related Aristolochia species constructed based on the psbA-trnH sequence alignment.

FIG. 5a shows a trnL-trnF sequence alignment of Fangji and related Aristolochia species.

FIG. 5b shows a dendrogram of Fangji and related Aristolochia species constructed based on the trnL-trnF sequence alignment.

FIG. 6a shows a psbA-trnH sequence alignment of Fangji and related Aristolochia species.

FIG. 6b shows a dendrogram of Fangji and related Aristolochia species constructed based on the psbA-trnH sequence alignment.

FIG. 7a shows a trnL-trnF sequence alignment of Baiying and related Aristolochia species.

FIG. 7b shows a dendrogram of Baiying and related Aristolochia species constructed based on the trnL-trnF sequence alignment.

FIG. 8a shows a psbA-trnH sequence alignment of Baiying and related Aristolochia species.

FIG. 8b shows a dendrogram of Baiying and related Aristolochia species constructed based on the psbA-trnH sequence alignment.

FIG. 9a shows a trnL-trnF sequence alignment of Madouling and related species.

FIG. 9b shows a dendrogram of Madouling and related species constructed based on the trnL-trnF sequence alignment.

FIG. 10a shows a psbA-trnH sequence alignment of Madouling and related species.

FIG. 10b shows a dendrogram of Madouling and related species constructed based on the psbA-trnH sequence alignment.

FIG. 11a shows a trnL-trnF sequence alignment of Zhushalian and related species.

FIG. 11b shows a dendrogram of Zhushalian and related species constructed based on the trnL-trnF sequence alignment.

FIG. 12a shows a psbA-trnH sequence alignment of Zhushalian and related species.

FIG. 12b shows a dendrogram of Zhushalian and related species constructed based on the psbA-trnH sequence alignment.

FIG. 13 shows a trnL-trnF sequence alignment of Aristolochia adulterants or alternatives and the genuine species.

FIG. 14 shows a psbA-trnH sequence alignment of Aristolochia adulterants or alternatives and the genuine species.

### DETAILED DESCRIPTION

Reference will now be made in detail to a number of particular embodiments of the invention as examples to facilitate the understanding of the present invention. Exemplary embodiments of the invention are described in detail, although it will be apparent to those skilled in the relevant art that some features that are not particularly important to an understanding of the invention may not be shown for the sake of clarity. On the other hand, details provided in connection with the particular embodiment are by example only, of which various changes and modifications may be effected by one skilled in the art without departing from the spirit or scope of the invention.

### EXAMPLE 1

### Muxiang

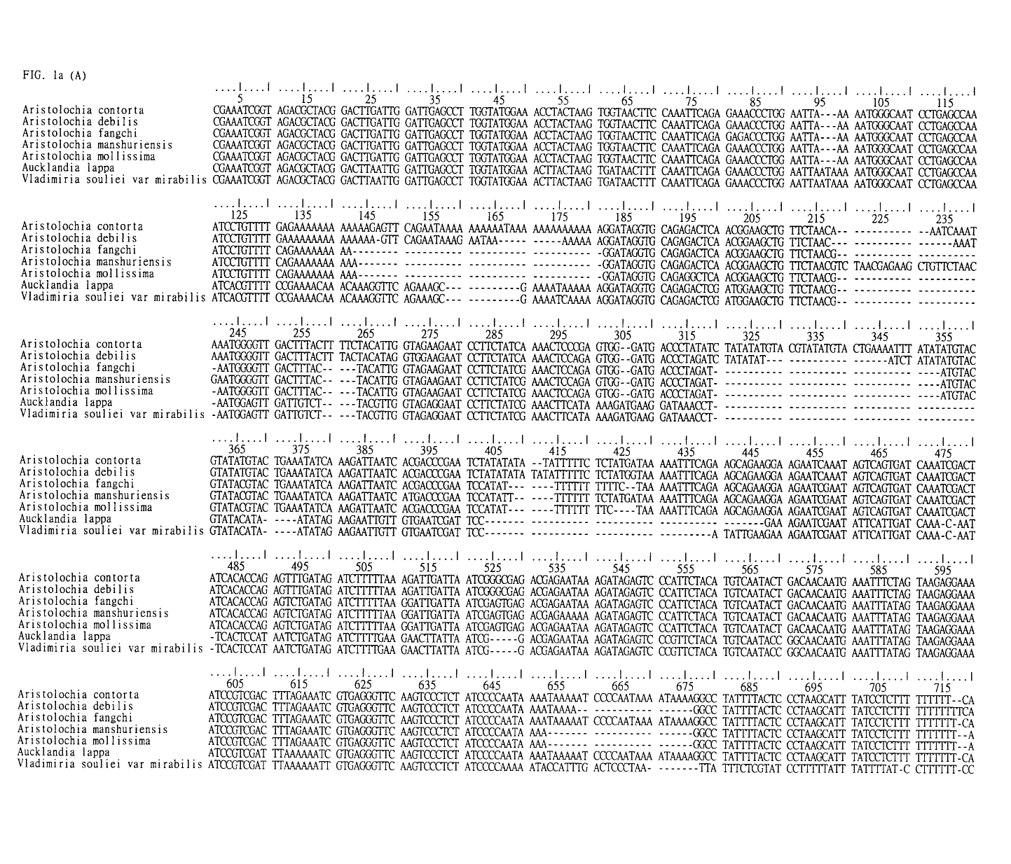

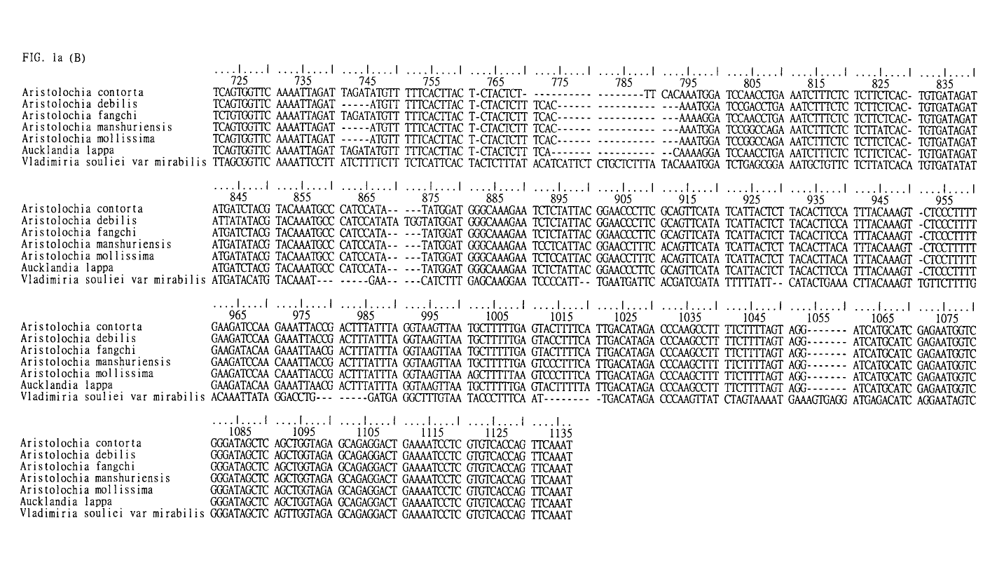

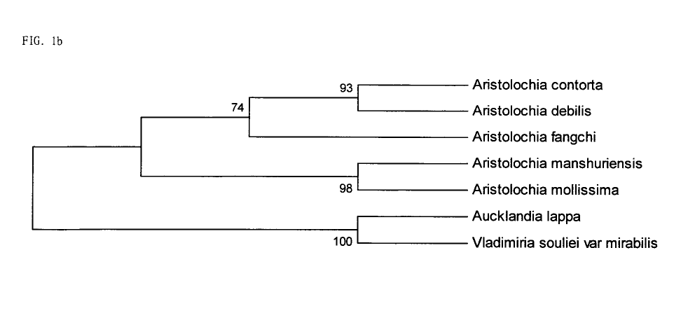

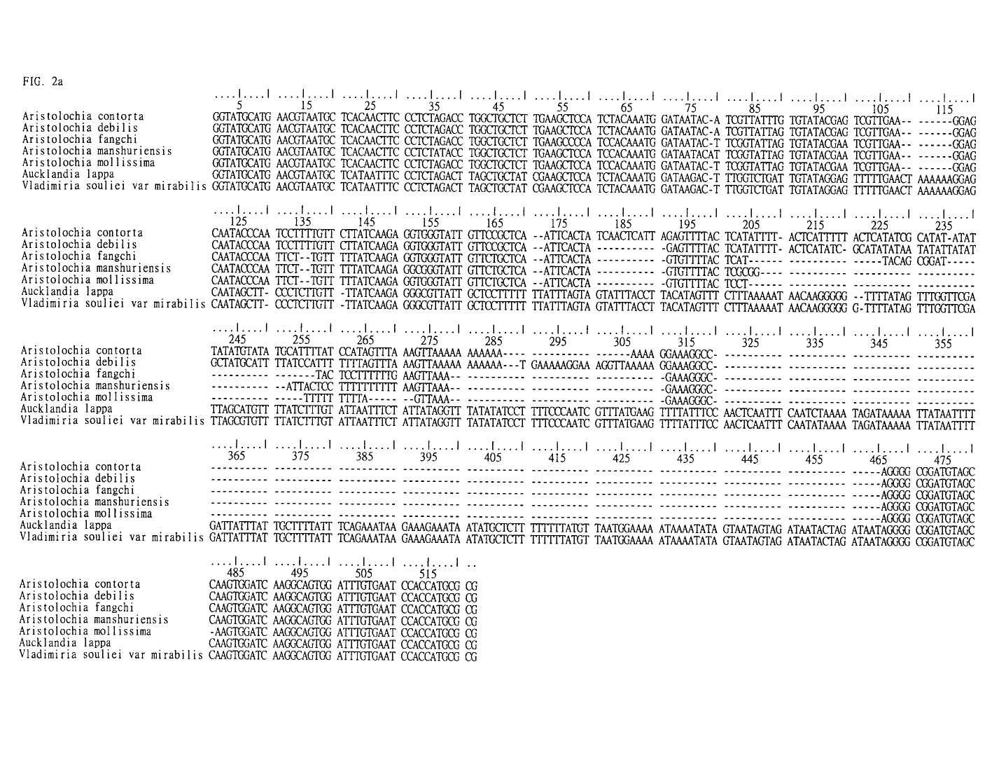

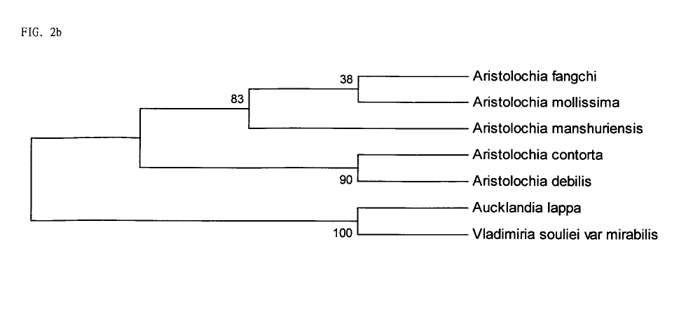

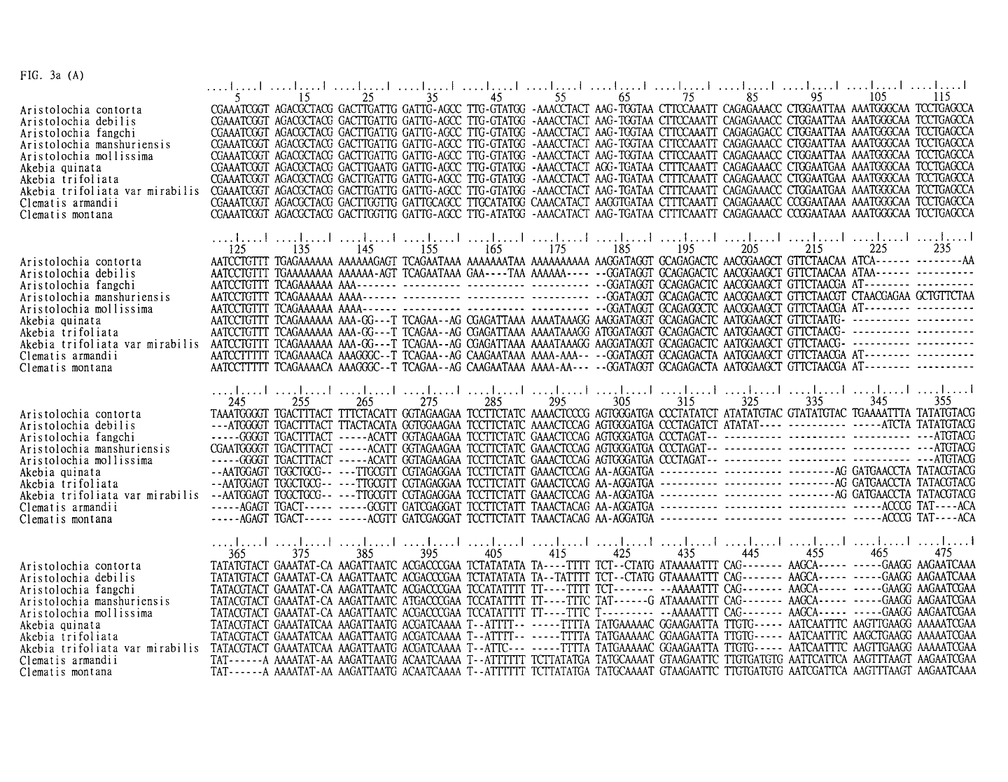

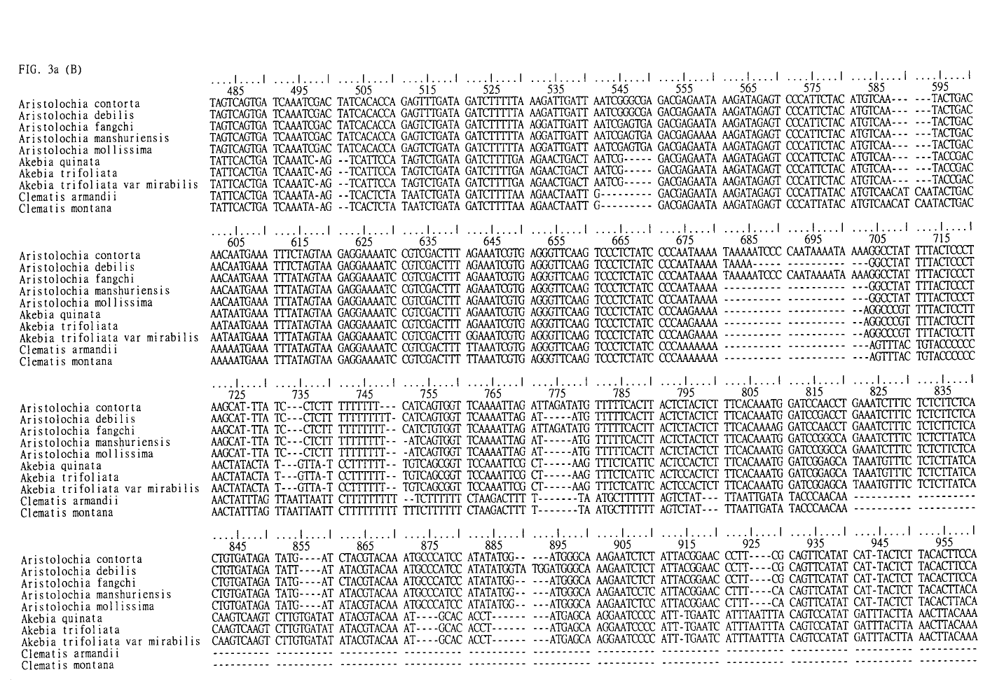

According to the Pharmacopoeia of the People's Republic of China (the 2005 edition), the Chinese herb Muxiang (or [symbol] in Chinese) is derived from Aucklandia lappa and some species of the daisy family (Compositae). It is sometimes adulterated by Aristolochia debilis, which is better known as Qingmuxiang (or [symbol] in Chinese) and contains the carcinogenic aristolochic acids. As shown in FIGS. 1 and 2, the trnL-trnF (FIGS. 1a and 1b) and psbA-trnH (FIG. 2a and 2b) sequences can be used to differentiate the genuine and adulterate species of Muxiang.

By aligning the Aristolochia trnL-trnF sequences with those of genuine Muxiang species (FIGS. 1a and 1b), it is clear that there are many differences; the more prominent ones are shown below:
(i) ‘TTAAAA’ in Aristolochia species but ‘TTMTAAAA’ in genuine Muxiang species (bases 93-101 in FIG. 1a), referred to as Genetic Marker 1;
(ii) ‘GTACTGAAATA’ in Aristolochia species but ‘ATMTA’ in genuine Muxiang species (bases 367-377 in FIG. 1a), referred to as Genetic Marker 2.

Similarly, aligning the Aristolochia psbA-trnH sequences with those of the genuine Muxiang species (FIGS. 2a and 2b), shows that there are many differences; the more prominent ones are shown below:
(i) ‘GMGGA’ in Aristolochia species but ‘GMCTAAAAAAGGA’ in genuine Muxiang species (bases 106-119 in FIG. 2a), referred to as Genetic Marker 3;
(ii) ‘CAGG’ in Aristolochia species but ‘CTAAAATAGATAAAAATTA TAATTTTGATTATTTATTGCTTTTATTTCAGAAATAAGAAAGA MTAATATGCTCTTTTTTTTATGTTMTGGAAAAATAAAATAT AGTAATAGTAGATAATACTAGATMTAGG’ in genuine Muxiang species (bases 335-468 in FIG. 2a), referred to as Genetic Marker 4.

### EXAMPLE 2

### Mutong

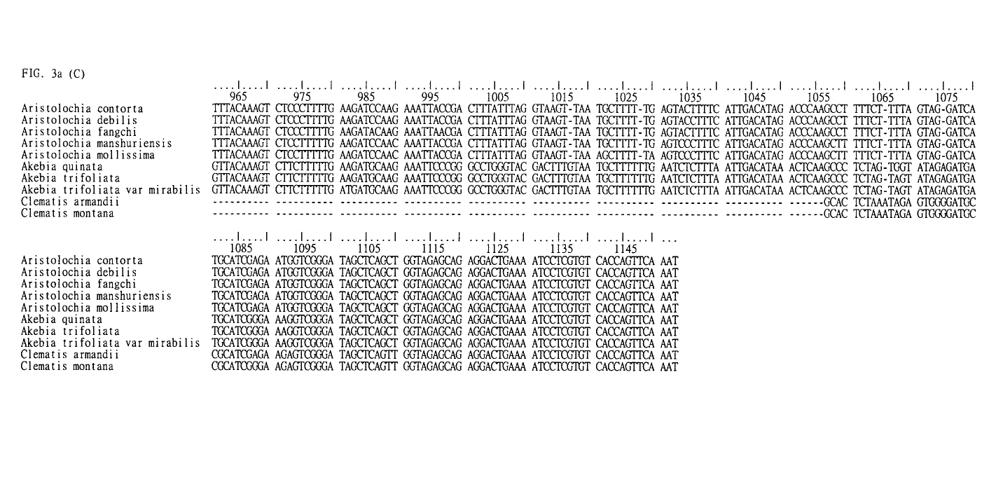

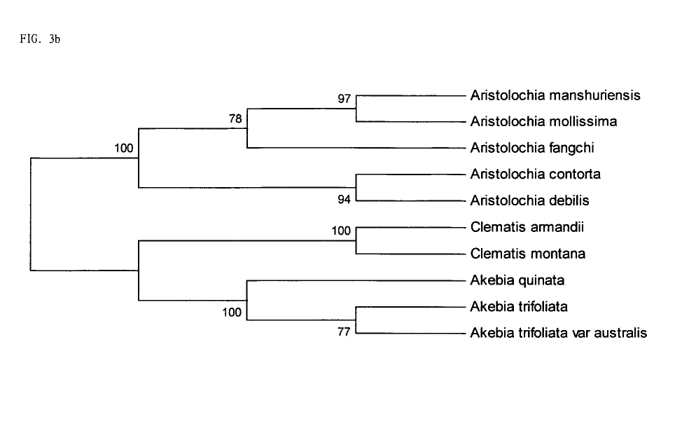

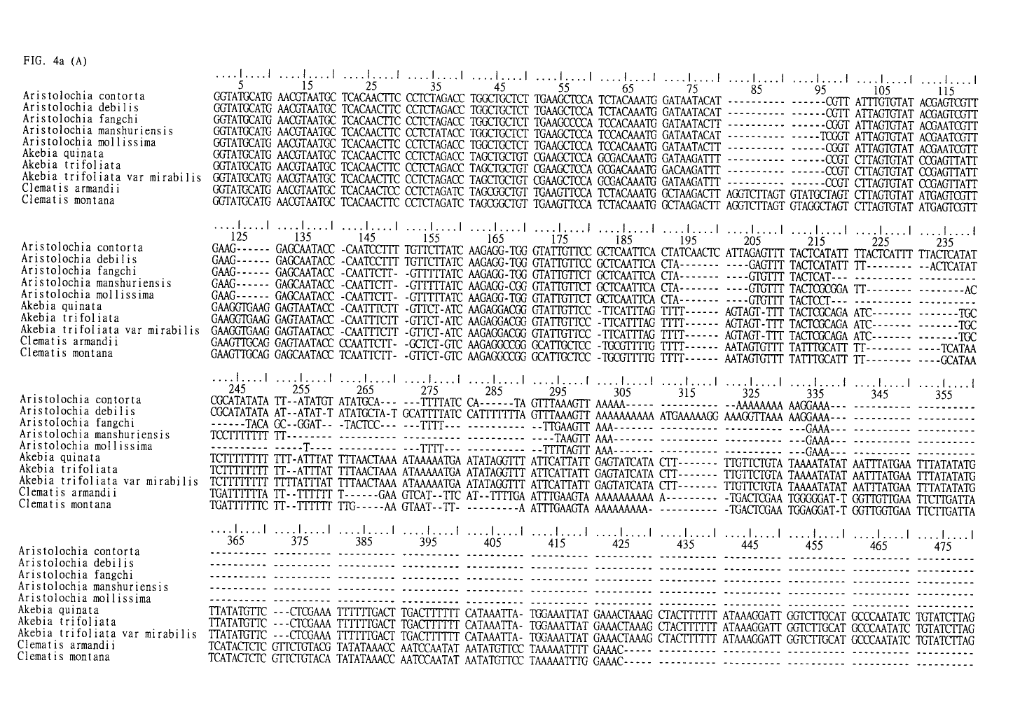

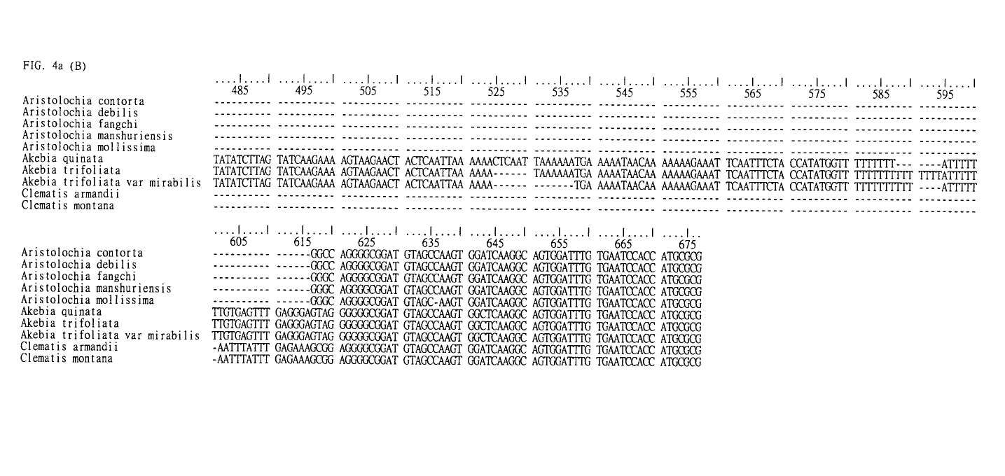

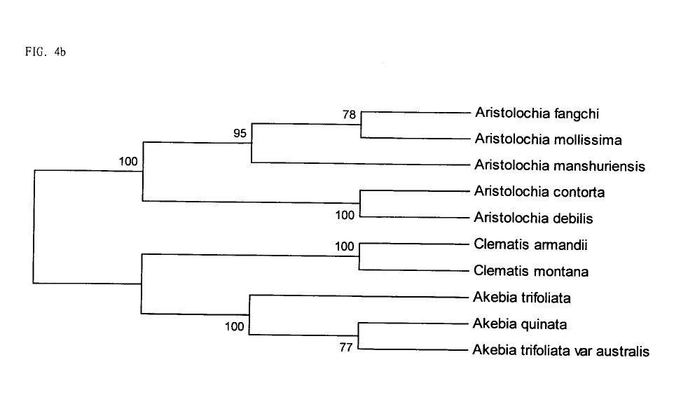

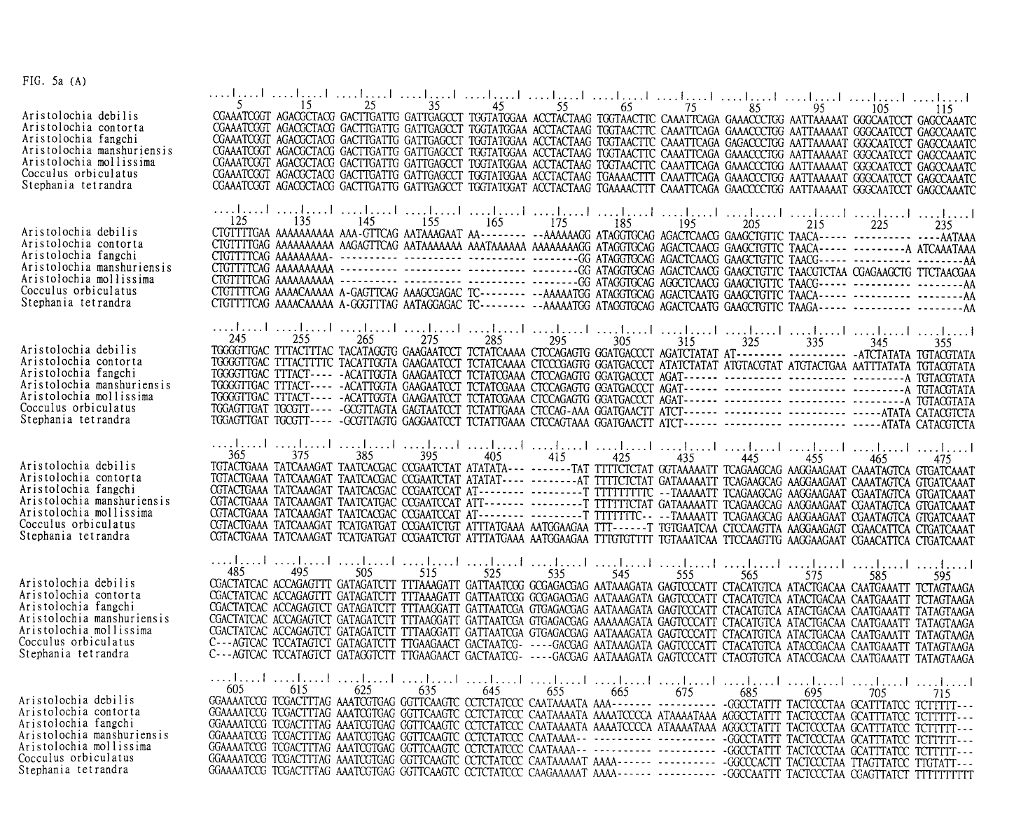

According to the Pharmacopoeia of the People's Republic of China (the 2005 edition), Mutong (or [symbol] in Chinese) is derived from Akebia quinata, Akebia trifoliata, Akebia trifoliata var. australis and may also include Chuanmutong (or [symbol] in Chinese), which is derived from Clematis armandii and Clematis montana. Mutong is sometimes adulterated by Guanmutong (Aristolochia manshuriensis), known as [symbol] in Chinese, which contains carcinogenic aristolochic acids. As shown in FIGS. 3 and 4, the trnL-trnF (FIGS. 3a and 3b) and psbA-trnH (FIGS. 4a and 4b) sequences can be used to differentiate the genuine and adulterate species of Mutong. Although the sequences of trnL-trnF region of Akebia quinata (Genbank AY651844; AF335297), Akebia trifoliata, (Genbank AF335294) and Aristolochia manshuriensis (Genbank AY689184), respectively, have been published for phylogenetic studies by three independent research groups (Quandt et al., 2004; Wang et al., 2002; Neinhuis et al., 2005), they have never been considered for any authentication purposes.

By aligning the Aristolochia trnL-trnF sequences with those of genuine Mutong species (FIGS. 3a and 3b), the differences are clear. The more prominent ones are shown below:
(i) ‘AGAGTGGGA’ in Aristolochia species but ‘AGAAAGGA’ in genuine Mutong species (bases 299-307 in FIG. 3a), referred to as Genetic Marker 5;
(ii) ‘GACTATCA’ in Aristolochia species but ‘AGTCA’ in genuine Mutong species (bases 498-505 in FIG. 3a), referred to as Genetic Marker 6.

Similarly, by aligning the Aristolochia psbA-trnH sequences with those of the genuine Mutong species (FIGS. 4a and 4b), it can be noted that among many differences, the more prominent ones are shown below:
(i) Absent in Aristolochia species but ‘GAAAC’ in genuine Mutong species (bases 421-425 in FIG. 4a), referred to as Genetic Marker 7;
(ii) Absent in Aristolochia species but ‘TTTGAG’ in genuine Mutong species (bases 608-613 in FIG. 4a), referred to as Genetic Marker 8.

### EXAMPLE 3

### Fangi

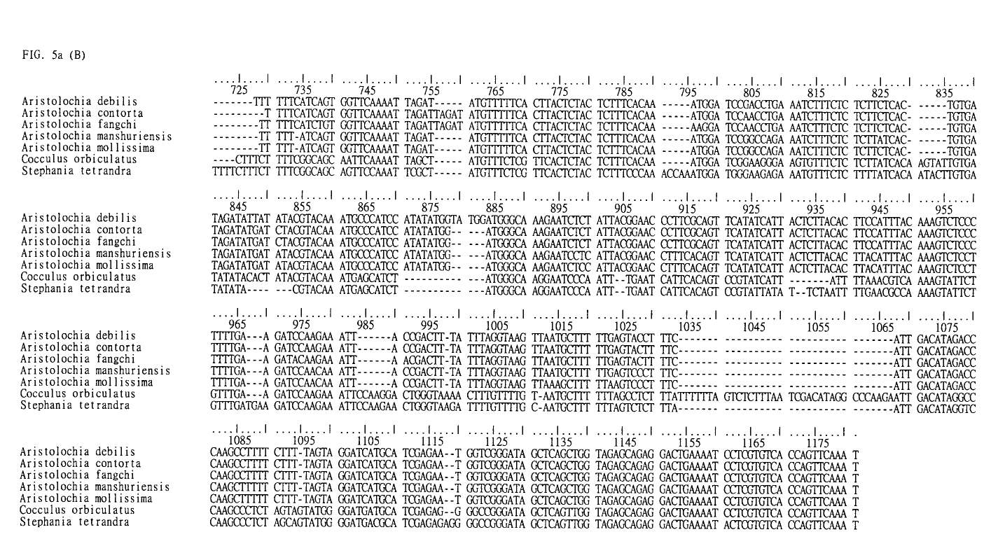

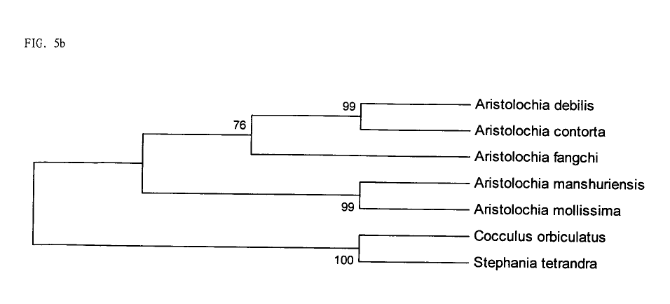

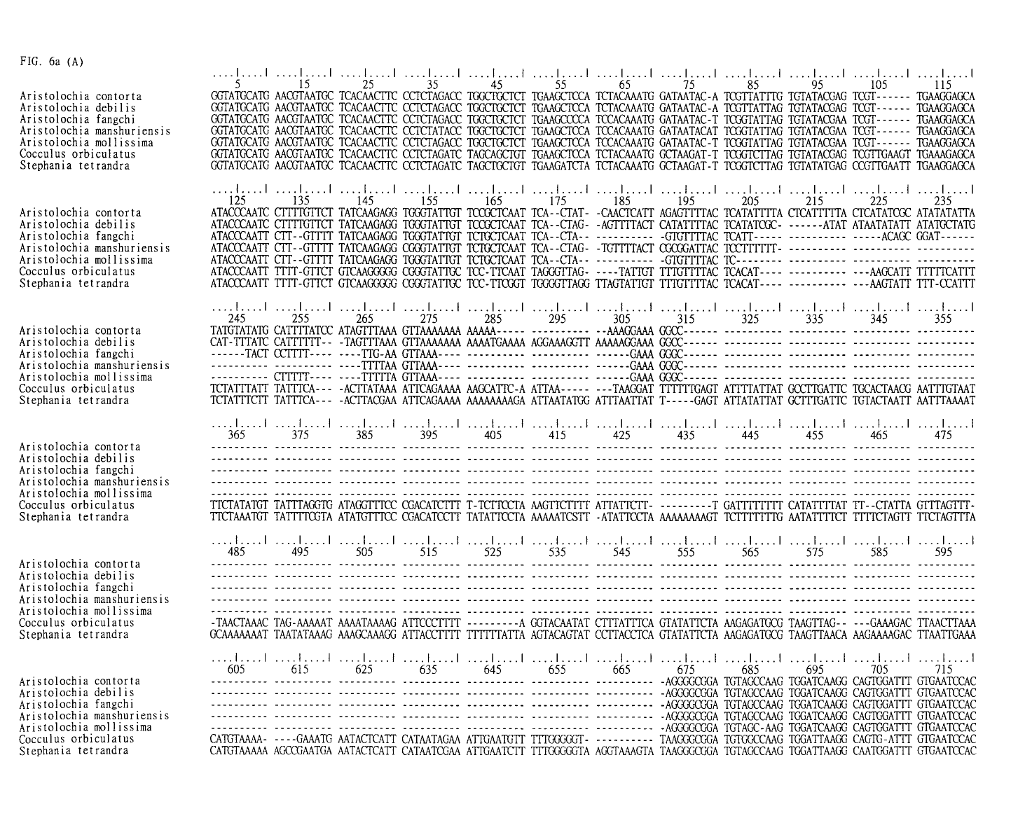

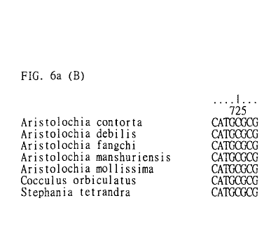

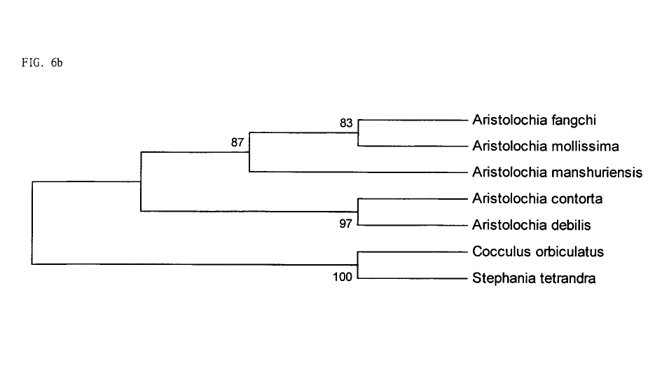

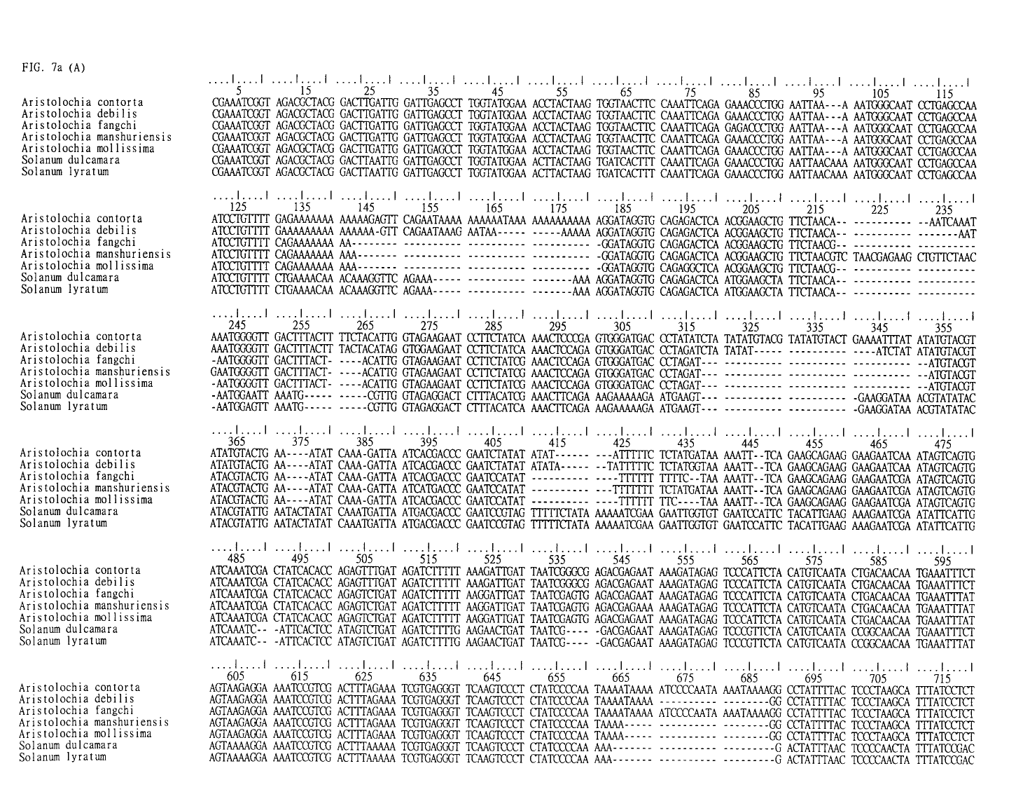

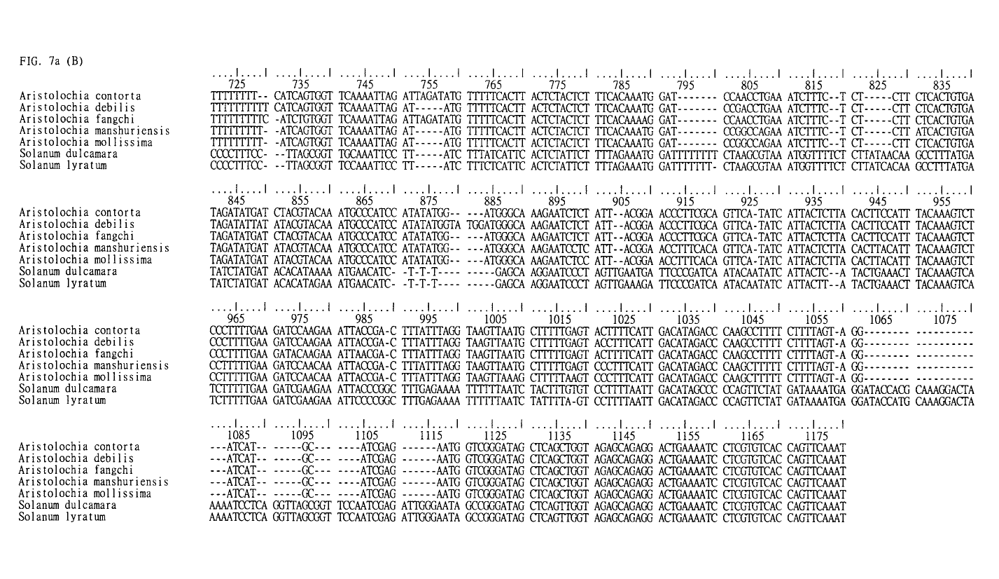

According to the Pharmacopoeia of the People's Republic of China (the 2005 edition), Fangji (or [symbol] in Chinese) is derived from Stephania tetrandra. It is sometimes adulterated by Guangfangji (Aristolochia fangchi and related Aristolochia species) referred to as [symbol] in Chinese, which contains carcinogenic aristolochic acids. It can also be confused with Mufangji (or [symbol] ” in Chinese) derived from Cocculus trilobus and related Cocculus species. As detailed in FIGS. 5 and 6, the trnL-trnF (FIGS. 5a and 5b) and psbA-trnH (FIGS. 6a and 6b) sequences can be used to differentiate Aristolochia species from other plant species used as Fangji.

By aligning the Aristolochia trnL-trnF sequences with those of genuine Fangji species (FIGS. 5a and 5b), it is clear that there are many differences; the more prominent ones are listed below:
(i) ‘GTGGGA’ in Aristolochia species but ‘AAAGGA’ in genuine Fangji species (bases 298-303 in FIG. 5a), referred to as Genetic Marker 9;
(ii) ‘ATCGACTATCA’ in Aristolochia species but ‘ATCAGTCA’ in genuine Fangji species (bases 479-489 in FIG. 5a), referred to as Genetic Marker 10;
(iii) ‘TTTTTCACTTACT’ in Aristolochia species but ‘TTTCTCGTTCA CT’ in genuine Fangji species (bases 764-776 in FIG. 5a), referred to as Genetic Marker 11;
(iv) ‘TCCATATATGG’ in Aristolochia species but ‘TCT’ in genuine Fangji species (bases 868-878 in FIG. 5a), referred to as Genetic Marker 12;
(v) ‘ATT’ in Aristolochia species but ‘ATTCCAAGG’ in genuine Fangji species (bases 981-988 in FIG. 5a), referred to as Genetic Marker 13;
(vi) ‘TAGTAGGA’ in Aristolochia species but ‘TATGGGGA’ in genuine Fangji species (bases 1096-1103 in FIG. 5a), referred to as Genetic Marker 14.

Similarly, by aligning the Aristolochia psbA-trnH sequences with those of the genuine Fangji species (FIGS. 6a and 6b), it becomes clear that there are many differences; the more prominent ones are listed below:
(i) ‘CGT’ in Aristolochia species but ‘CGTTGAA’ in genuine Fangji species (bases 102-108 in FIG. 6a), referred to as Genetic Marker 15;

1(ii) Absent in Aristolochia species but ‘GTTTCCCGACATC’ in genuine Fangji species (bases 385-397 in FIG. 6a), referred to as Genetic Marker 16.

### EXAMPLE 4

### Baiying

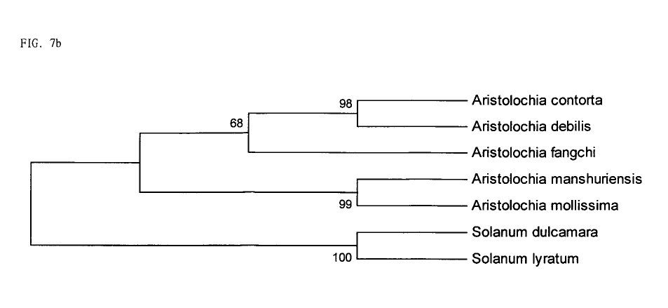

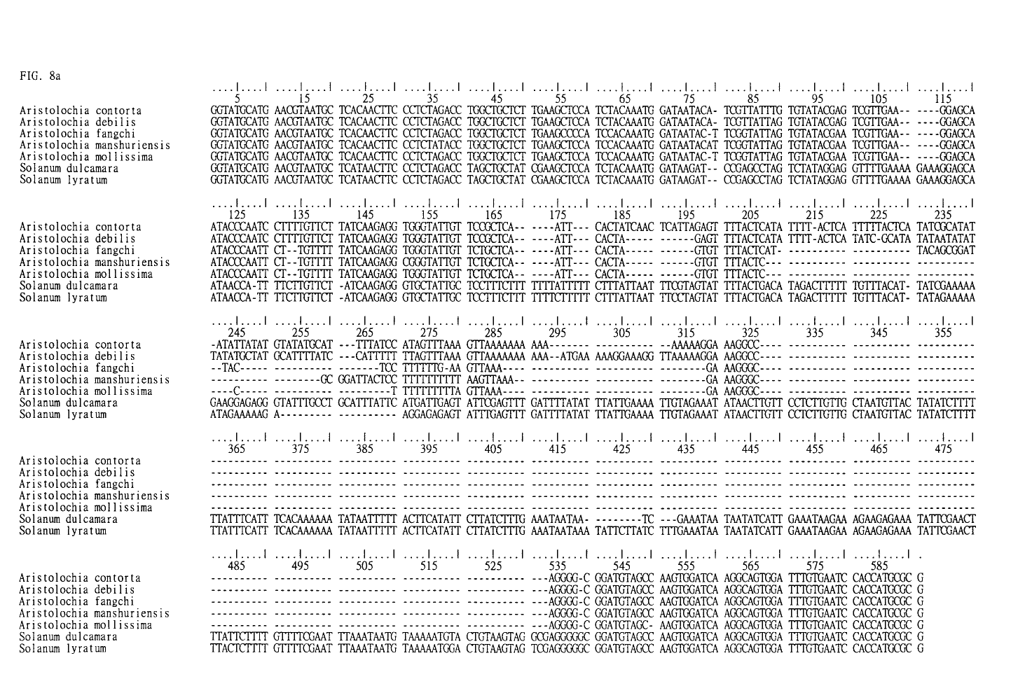

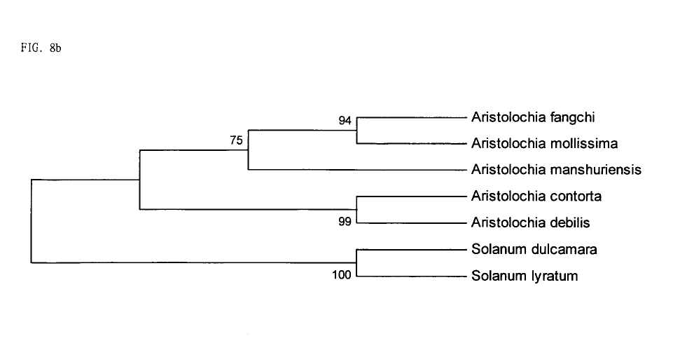

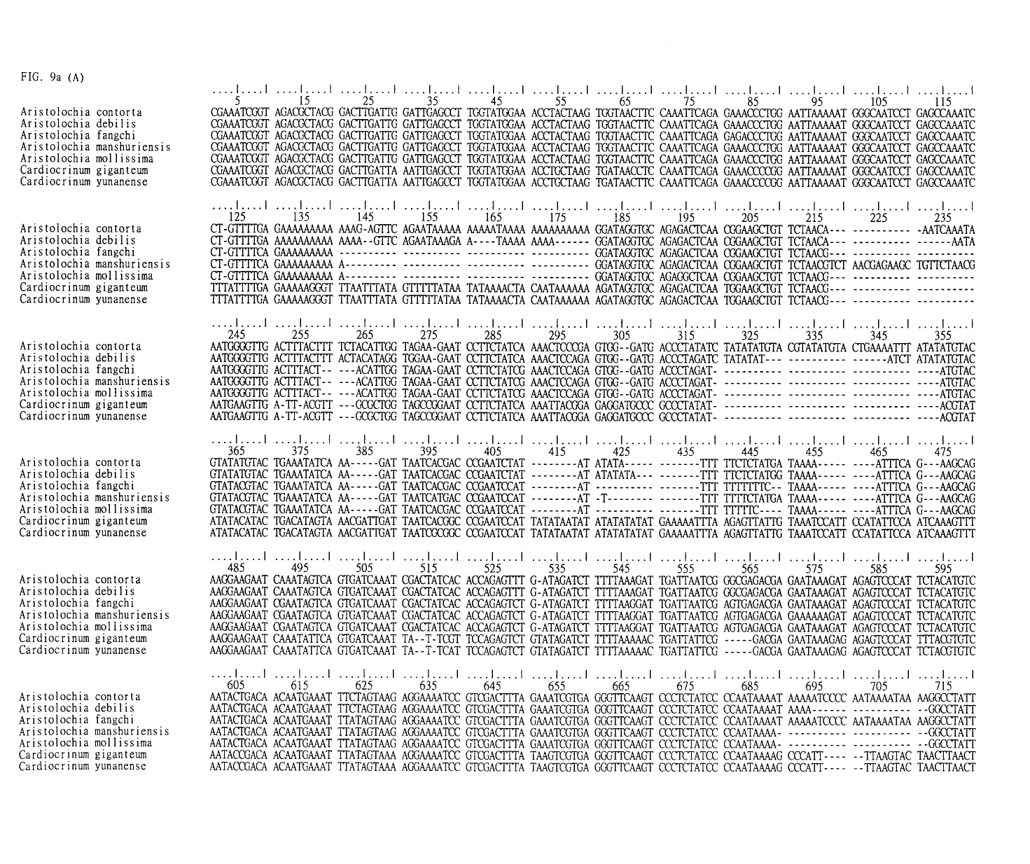

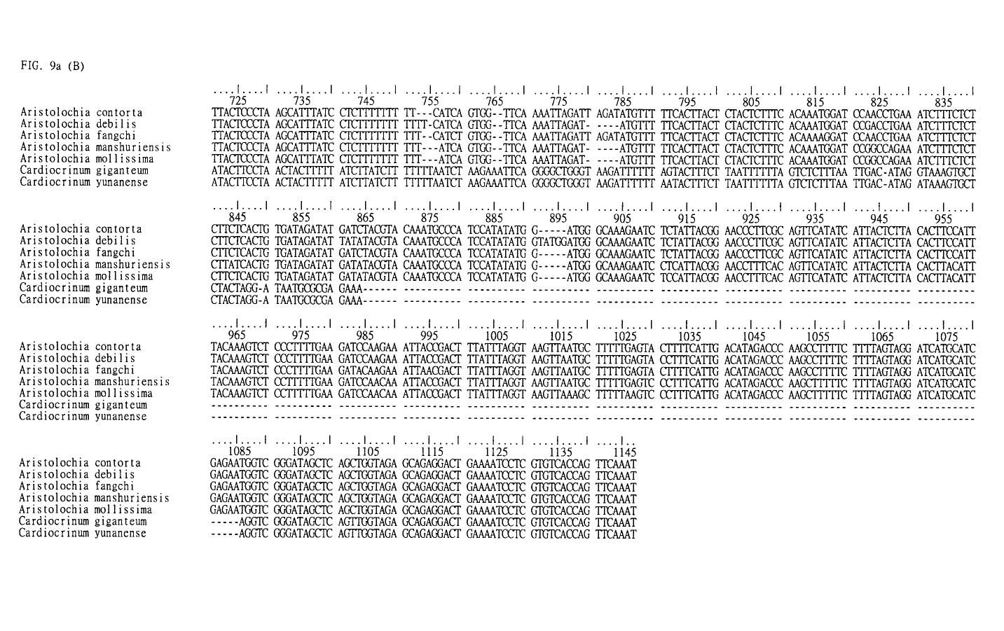

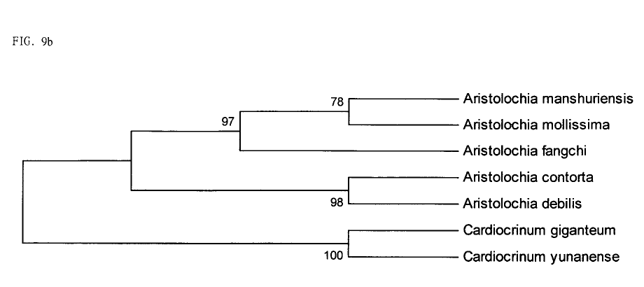

According to the Pharmacopoeia of the People's Republic of China (1977 edition), Baiying (or [symbol] in Chinese) is derived from Solanum lyratum. It is sometimes adulterated by Xungufeng (Aristolochia mollissima), known as [symbol] in Chinese, which contains carcinogenic aristolochic acids. As detailed in FIGS. 7 and 8, the trnL-trnF (FIGS. 7a and 7b) and psbA-trnH (FIGS. 8a and 8b) sequences can be used to differentiate the genuine from the adulterate species of Baiying.

Aligning the Aristolochia trnL-trnF sequences with those of genuine Baiying species (FIGS. 7a and 7b), reveals that there are many differences; the more prominent ones are listed below:
(i) ‘TAAAAA’ in Aristolochia species but ‘TAACAAAAA’ in genuine Baiying species (bases 94-102 in FIG. 7a), referred to as Genetic Marker 17;
(ii) ‘GAAATA’ in Aristolochia species but ‘GAATACTATA’ in genuine Baiying species (bases 370-379 in FIG. 7a), referred to as Genetic Marker 18;
(iii) ‘ACTGTG’ in Aristolochia species but ‘ACAAGCCTTTATG’ in genuine Baiying species (bases 827-839 in FIG. 7a), referred to as Genetic Marker 19;
(iv) ‘TTTATTTAGGTAAGTTA’ in Aristolochia species but ‘TTTGAGAAAATTTTTTA’ in genuine Baiying species (bases 991-1007 in FIG. 7a), referred to as Genetic Marker 20;
(v) ‘GAGAAT’ bsent in Aristolochia species but ‘GAGATTGGAAT’ in genuine Baiying species (bases 1108-1119 in FIG. 7a), referred to as Genetic Marker 21.

Similarly, by aligning the Aristolochia psbA-trnH sequences with those of the genuine Baiying derived from Solanum species (FIGS. 8a and 8b), it is clear that there are many differences; the more prominent ones are listed below:
(i) ‘GAAGGA’ bsent in Aristolochia species but ‘GAAAAGAAAGG A’ in genuine Baiying species (bases 106-117 in FIG. 8a), referred to as Genetic Marker 22;
(ii) Absent in Aristolochia species but ‘TGTTCCTCTTGTTGCTAAT GTTACTATATCTTTTTTATTTCATTTCACAAAAAATATAATTTT TACTTCATATTCTTATCTTTGAAATAATAA’ in genuine Baiying species (bases 327-419 in FIG. 8a), referred to as Genetic Marker 23;
(iii) Absent in Aristolochia species but ‘AAATAATAATATCATTGAA ATAAGAAAGAAGAGAAATATTCGAACTTTA’ in genuine Baiying species (bases 434-483 in FIG. 8a), referred to as Genetic Marker 24.

### EXAMPLE 5

### Madouling

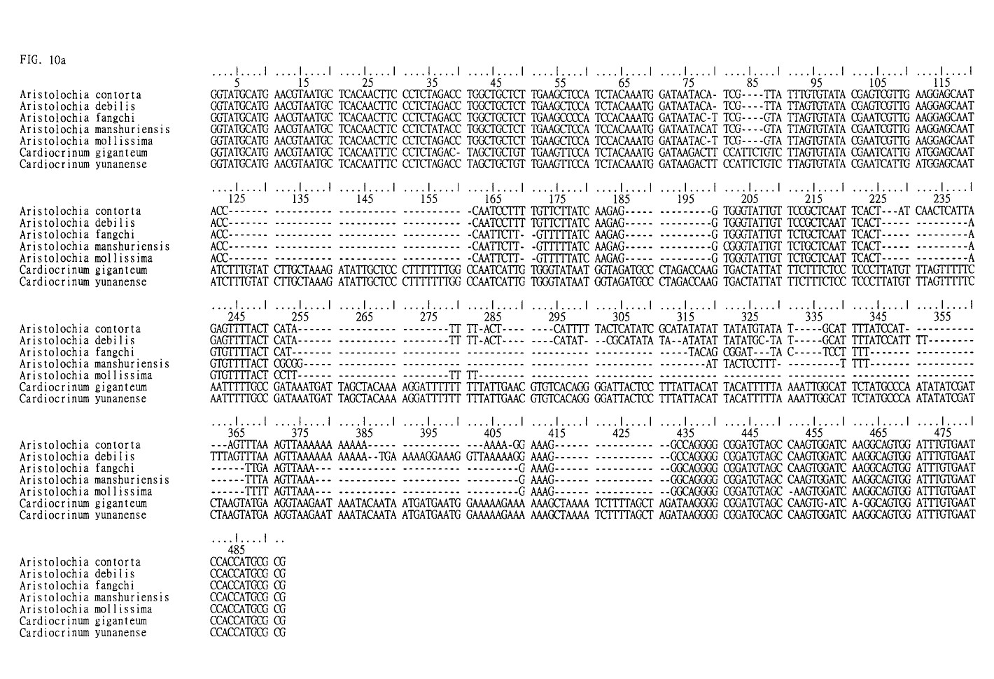

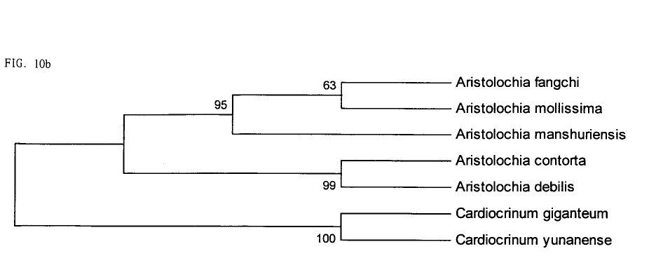

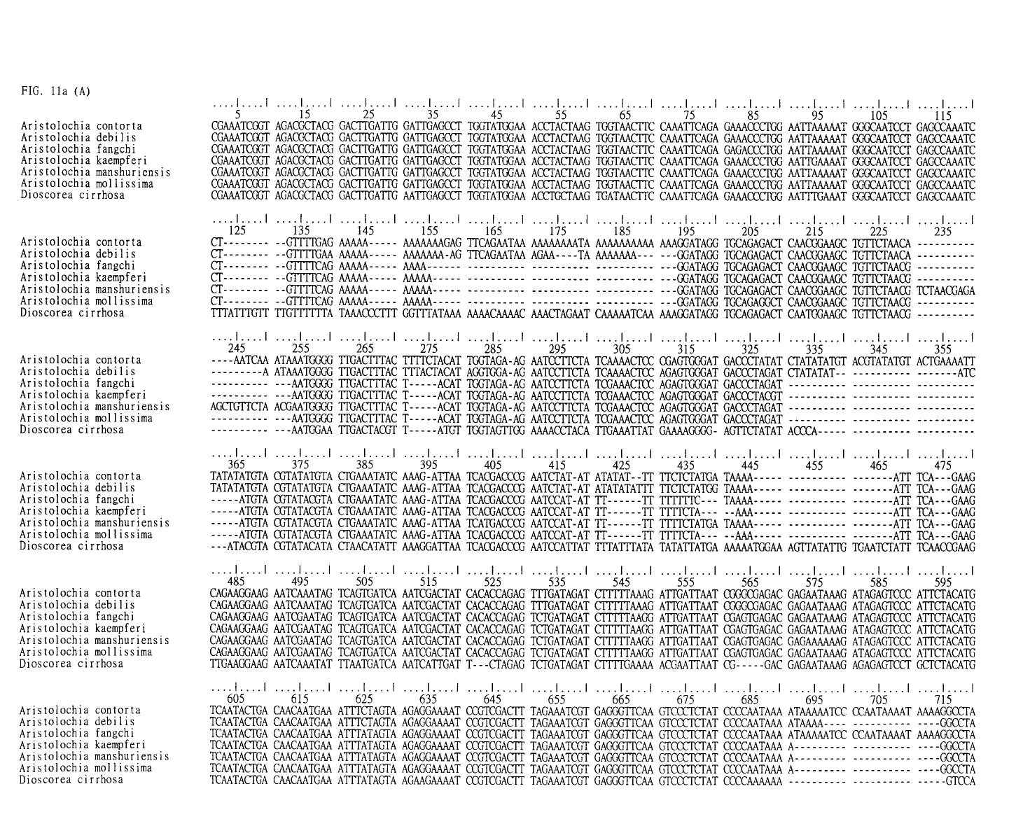

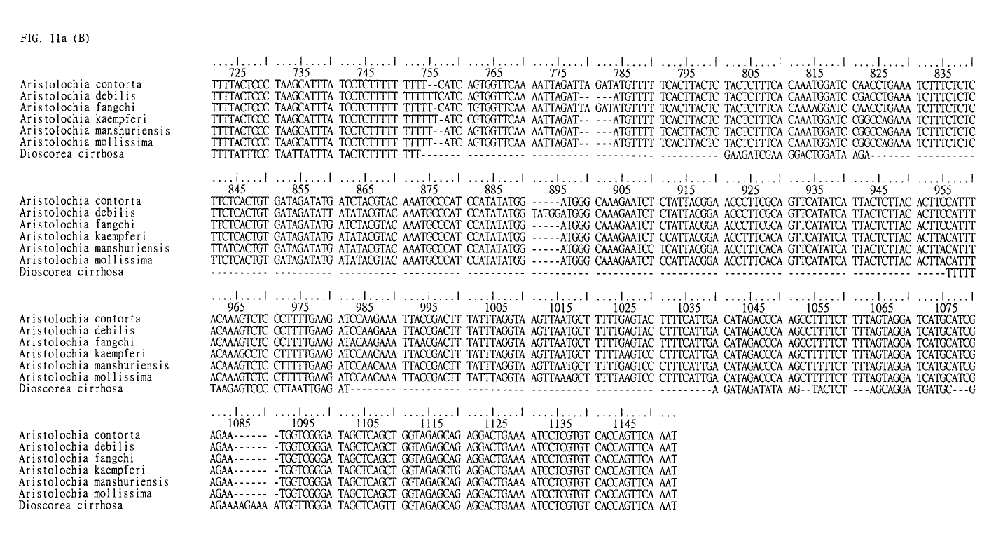

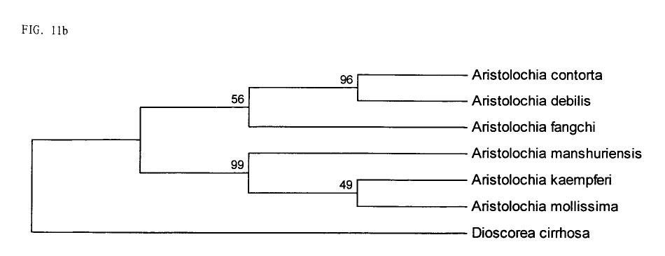

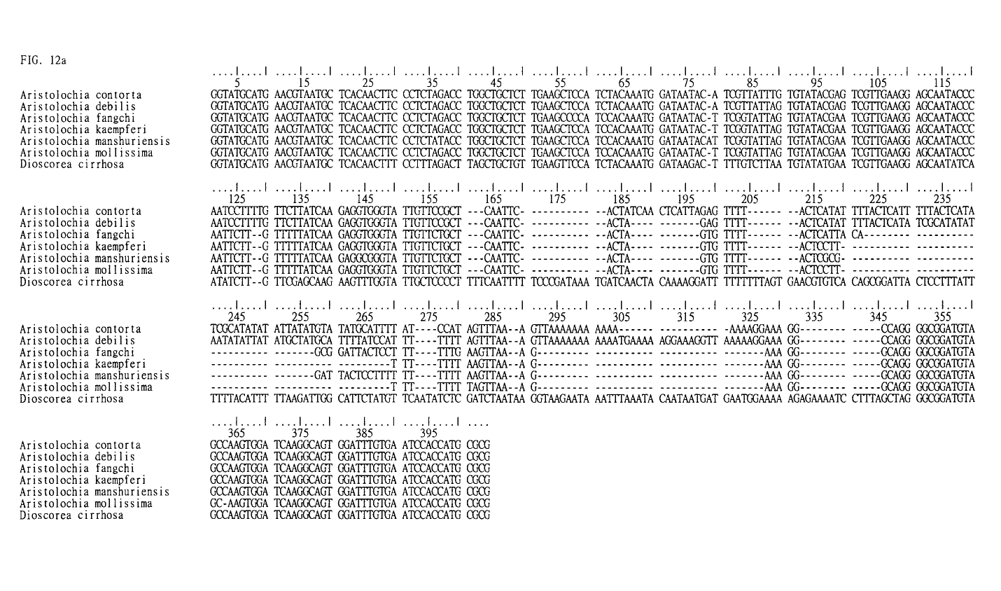

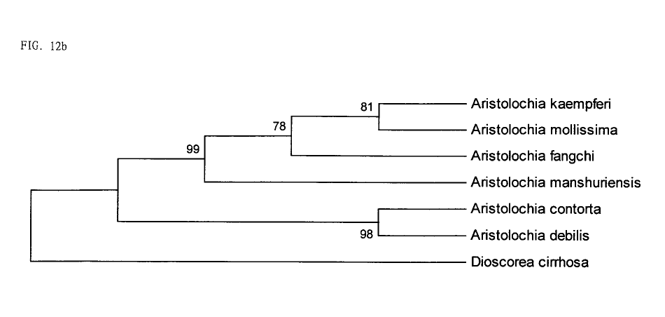

According to the Pharmacopoeia of the People's Republic of China (the 2005 edition), Madouling (or [symbol] in Chinese) is derived from Aristolochia contorta and Aristolochia debilis, which contain carcinogenic aristolochic acids and thus must be used more carefully. Its alternatives include Dabaihe, which is known as [symbol] in Chinese and is derived from Cardiocrinum giganteum and related Liliaceae plants. The trnL-trnF (FIGS. 9a and 9b) and psbA-trnH (FIGS. 10a and 10b) sequences can used to differentiate the genuine and alternative species of Madouling.

By aligning the Aristolochia trnL-trnF sequences with those of Dabaihe species (FIGS. 9a and 9b), it is clear that there are many differences; the more prominent ones are listed below:
(i) ‘TTTCACAAA’ in Aristolochia species but ‘TTTAGTCTC’ in Dabaihe species (bases 807-815 in FIG. 9a), referred to as Genetic Marker 25;
(ii) ‘TACGTACAAATGCCCATCCATATATGG’ in Aristolochia species but absent in Dabaihe species (bases 865-891 in FIG. 9a), referred to as Genetic Marker 26.

Similarly, by aligning the Aristolochia psbA-trnH sequences with those of Dabaihe species (FIGS. 10a and 10b), it becomes clear that there are many differences; the more prominent ones are listed below:
(i) ‘CG’ in Aristolochia species but ‘CATTCT’ in Dabaihe species (bases 82-87 in FIG. 10a), referred to as Genetic Marker 27;
(ii) ‘TACCCAA’ in Aristolochia species but ‘TACCTTTGTATCTTG CTAAAGATATTGCTCCCTTTTTTTGGCCAA’ in Dabaihe species (bases 120-164 in FIG. 10a), referred to as Genetic Marker 28;
(iii) ‘AAGG’ in Aristolochia species but ‘AAGCTAAAATCTTTT AGCTAGA’ in Dabaihe species (bases 412-433 in FIG. 10a), referred to as Genetic Marker 29.

### EXAMPLE 6

### Zhushalian

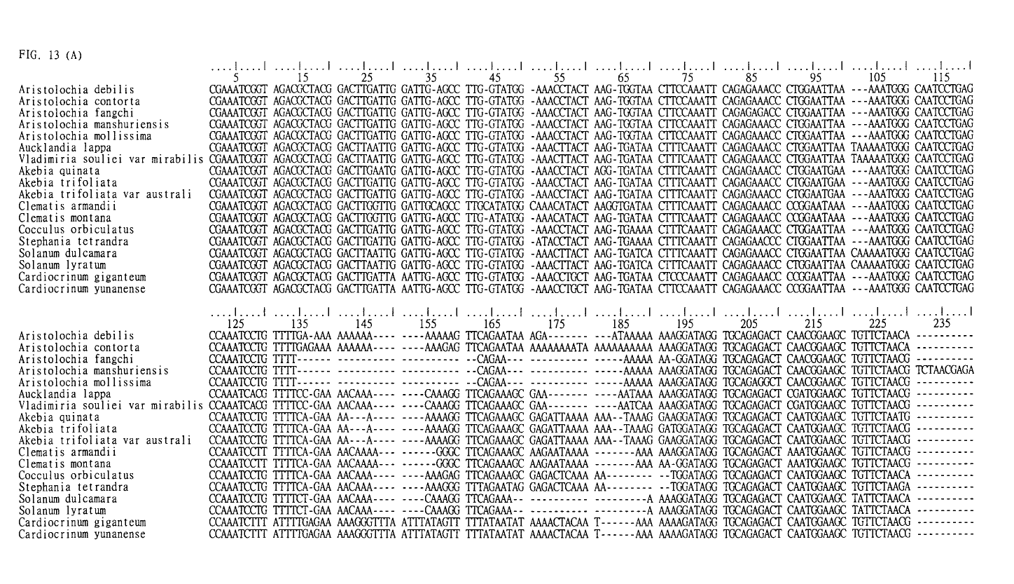

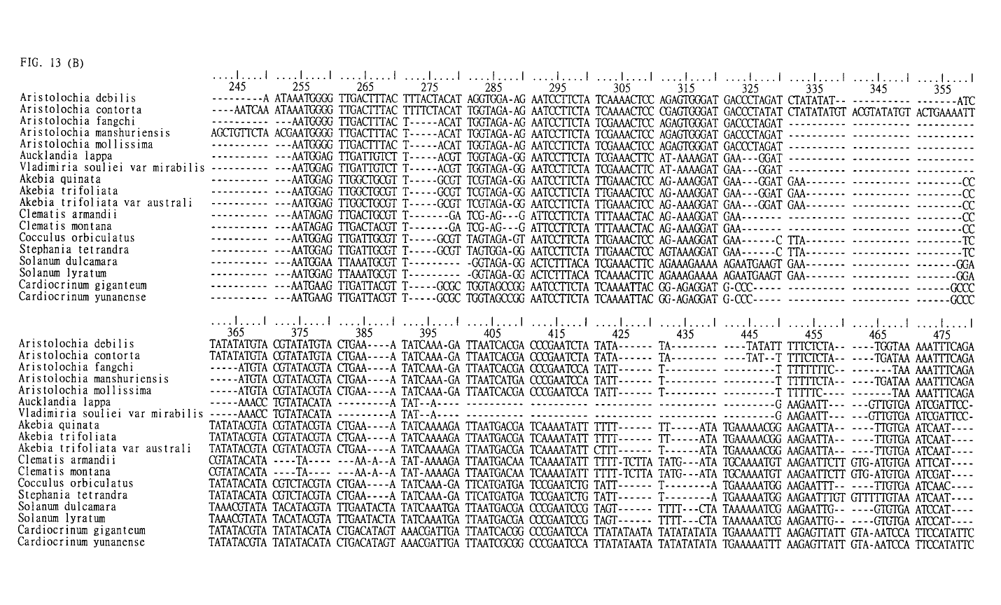

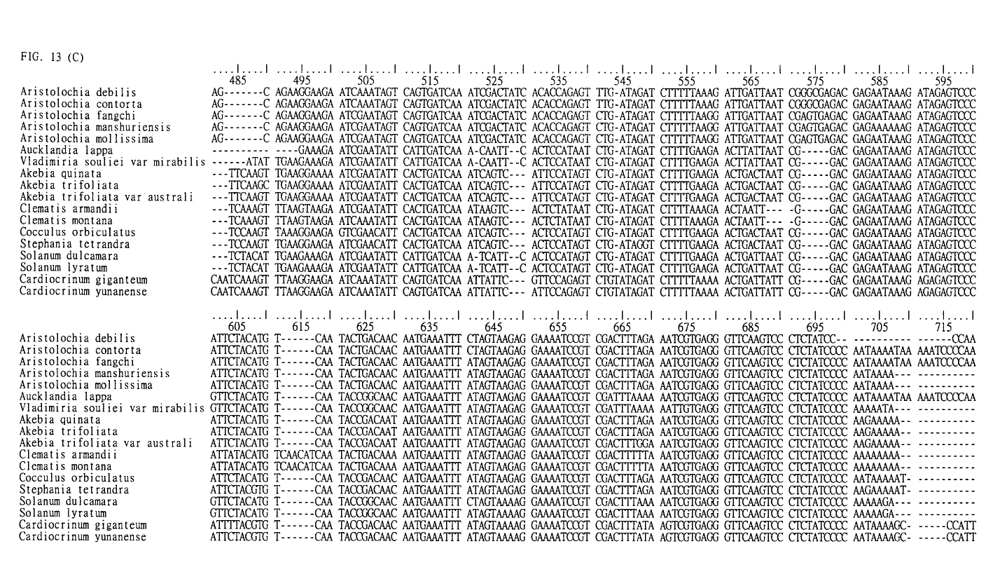

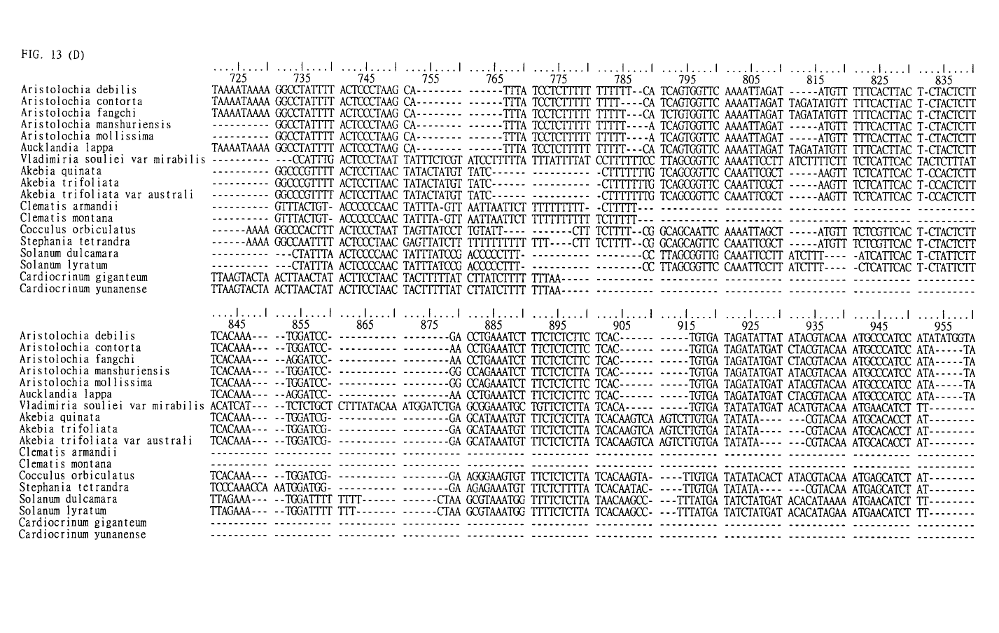

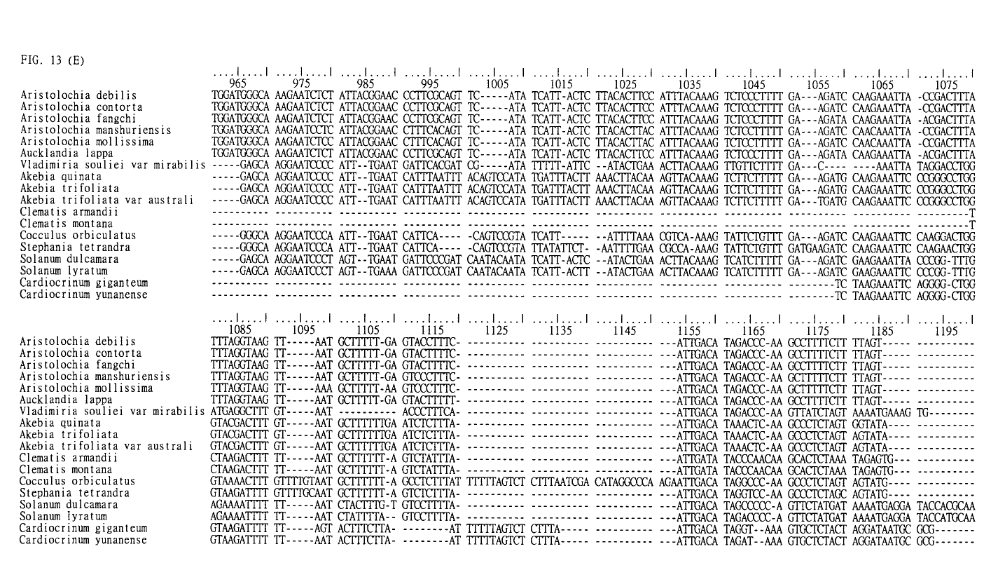

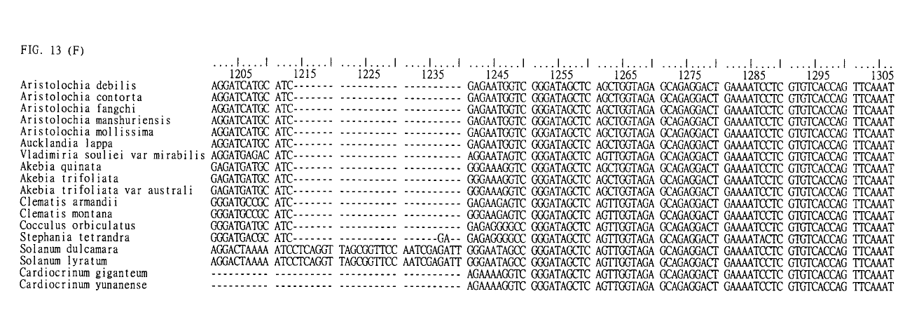

According to GuanMuTongShenDuXingYanJiu [symbol] Chinese) Zhushalian (or [symbol] in Chinese) is derived from Aristolochia kaempferi and related species. Its alternatives include roots derived from Dioscorea cirrhosa, known as Shuliang (or [symbol] in Chinese), and related species in the family Dioscoreaceae. The trnL-trnF (FIGS. 11a and 11b) and psbA-trnH (FIGS. 12a and 12b) sequences can used to differentiate the genuine and alternative species of Zhushalian.

By aligning the Aristolochia trnL-trnF sequences with those of Shuliang species (FIGS. 11a and 11 b), it is clear that there are many differences; the more prominent ones are listed below:
(iii) ‘ATCCTGTT’ in Aristolochia species but ‘ATCTTTATTTGTT TTGTT’ in Shuliang species (bases 118-135 in FIG. 11a), referred to as Genetic Marker 30;
(iv) ‘TCAGAAGCAGA’ in Aristolochia species but ‘TCAACCGA AGTTGA’ in Shuliang species (bases 471-484 in FIG. 11a), referred to as Genetic Marker 31;
(v) ‘ATCGACTATCACACCAGA’ in Aristolochia species but ‘ATCATTGATTCTAGA’ in Shuliang species (bases 512-529 in FIG. 11a), referred to as Genetic Marker 32;
(vi) ‘TGCATCGAGAATGGTCGG’ in Aristolochia species but ‘TGCGAGAAAAGAAAATGGTTGGG’ in Shuliang species (bases 1074-1098 in FIG. 11a), referred to as Genetic Marker 33.

Similarly, by aligning the Aristolochia psbA-trnH sequences with those of Shuliang (FIGS. 12a and 12b), it is clear that there are many differences; the more prominent ones are listed below:
(iv) ‘ACCTGGC’ in Aristolochia species but ‘ACTTAGC’ in Shuliang species (bases 38-44 in FIG. 12a), referred to as Genetic Marker 34;
(v) ‘ATACCCAAT’ in Aristolochia species but ‘ATATCAATA’ in Shuliang species (bases 115-123 in FIG. 12a), referred to as Genetic Marker 35.

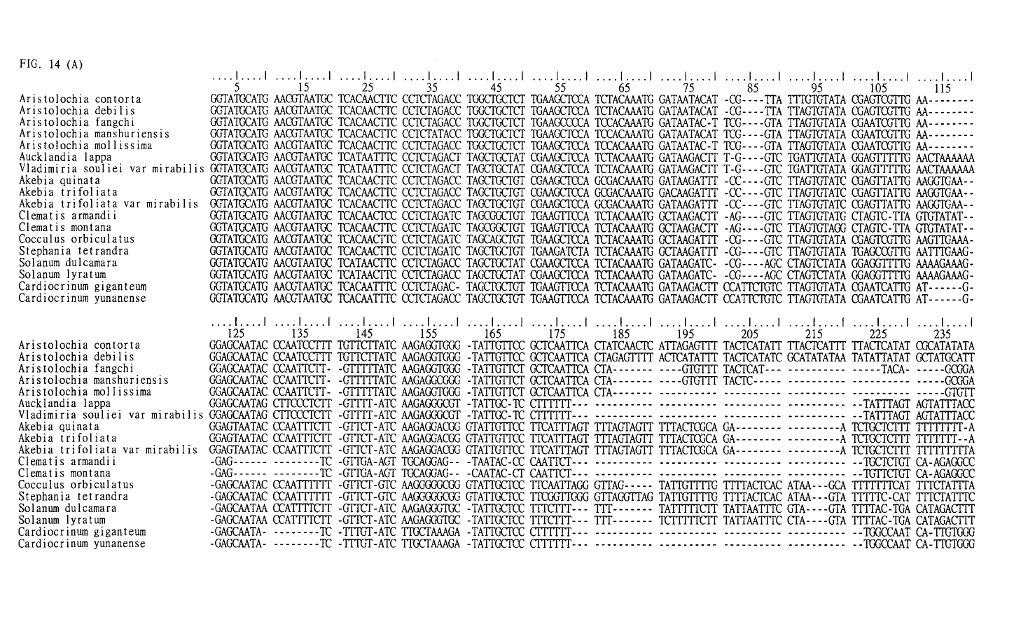

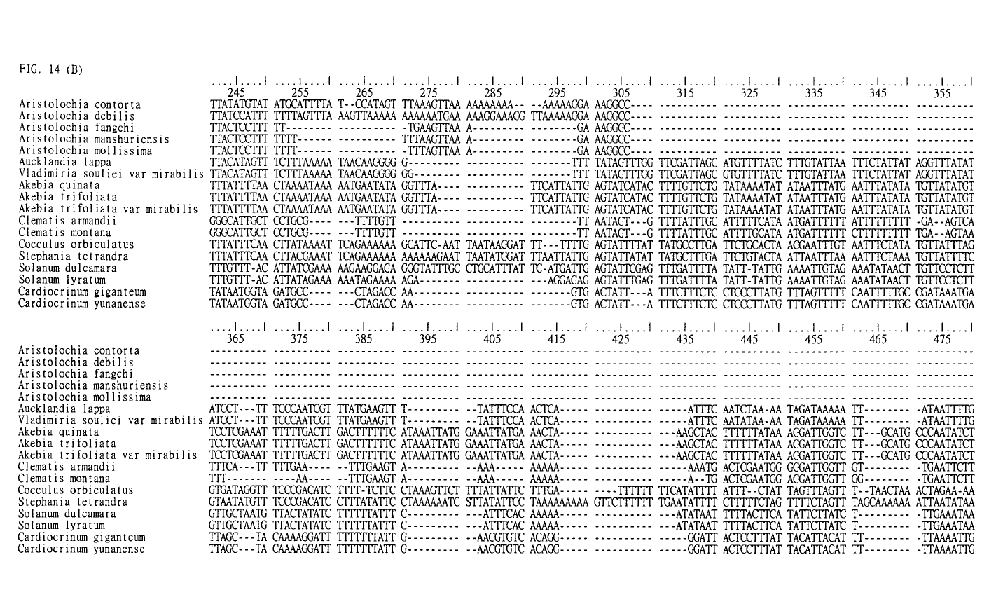

When the sequences of all the herbs in the above five examples are aligned together, it is noted that, in general, the Aristolochia trnL-trnF region has specific sequence segments which are characteristic of the plants of the Aristolochia genus when compared with the other plant species of Muxiang, Mutong, Fangji, Baiying and Dabaihe (FIG. 13). Some examples of the characteristic segments are as follows:
(i) ‘AGTGGGATG’ in Aristolochia species (bases 313-321 in FIG. 11), referred to as Genetic Marker 36;
(ii) ‘AATTTCAGAAG’ in Aristolochia species (bases 472-482 in FIG. 11), referred to as Genetic Marker 37;
(iii) ‘ACTATCACACC’ in Aristolochia species (bases 525-535 FIG. 11), referred to as Genetic Marker 38.

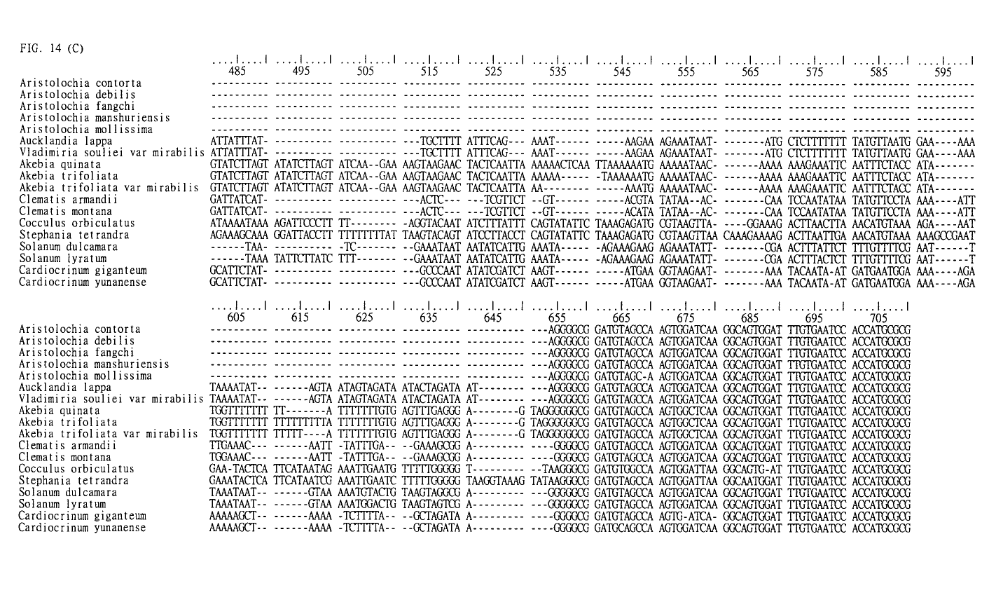

Similarly, the Aristolochia psbA-trnH region also has some specific segments which are characteristic of plants of the Aristolochia genus compared with the other plant species Muxiang, Mutong, Fangji, Baiying and Dabaihe (FIG. 14). An example is:
(i) ‘CTCAATTCACTA’ in Aristolochia species (bases 172-183 in FIG. 12), referred to as Genetic Marker 39.

In the above, each genetic marker (i.e., of Genetic Markers 1-39) comprises a number of sequences, each for a particular species as defined and aligned together in the corresponding FIGS. The sequences of each genetic marker are of the same or different length, also detailed in the corresponding FIGS.

The method of the present invention as exemplified in the foregoing examples can be utilized in many circumstances to determine whether Aristolochia herbs are present in botanicals or herbal products by making an extract of the total DNA from the samples of concern, followed by amplification of either or both of the trnL-trnF and psbA-trnH regions by polymerase chain reaction (PCR) and then by DNA sequencing. By comparing and matching the resulting trnL-trnF and psbA-trnH sequences to those of the Aristolochia herbs, the matched samples would belong to Aristolochia. As demonstrated in the foregoing examples, there are a number of sequence segments within the trnL-trnF and psbA-trnH regions that are characteristic of the plants of the Aristolochia genus. For particular cases, it may not be necessary to amplify and sequence the entire trnL-trnF region and/or the entire psbA-trnH region, but amplifying and sequencing a portion of the trnL-trnF region and/or the psbA-trnH region may be sufficient for a particular purpose in a particular situation.

For PCR and DNA sequencing of the trnL-trnF region, the forward primer is Tab C (5′-CGAAATCGGTAGACGCTACG-3′), and the reverse primer is Tab F (5′-ATTTGAACTGGTGACACGAG-3′). For PCR and DNA sequencing of psbA-trnH region, the forward primer is (5′-GGTATGCATGMCGTMTGCTC-3′), and the reverse primer is (5′-CGCGCATGGTGGATTCACAAATC-3′).

While there have been described and pointed out fundamental novel features of the invention as applied to preferred embodiments thereof, it will be understood that various omissions and substitutions and changes, in the form and details of the processes and methods illustrated, may be made by those skilled in the art without departing from the spirit of the invention. For example, it is expressly intended that all combinations of those elements of method steps which perform substantially the same function in substantially the same way to achieve the same results are within the scope of the invention.

The invention is not limited by the embodiments described above which are presented as examples only but can be modified in various ways within the scope of protection defined by the appended patent claims.

### REFERENCES

Arlt, V. M.; Stiborova, M.; Schmeiser, H. H. (2002) Aristolochia acid as a probable human cancer hazard in herbal remedies: a review. Mutagenesis, 17 (4): 265-277.
Bayer, R. J.; Starr, J. R. (1998) Tribal phylogeny of the Asteraceae based on two non-coding chloroplast sequences, the trnL intron and trnL/trnF intergenic spacer. Annals of the Missouri Botanical Garden, 85 (2): 242-256.
Hall, T. A. (1999) BioEdit: a user-friendly biological sequence alignment editor and analysis program for Windows 95/98/NT. Nucleic Acids Symposium. 41:95-98.
Kohjyouma, M.; Lee, I. J.; Iida, O.; Kurihara, K.; Yamada, K.; Makino, Y.; Sekita, S.; Satake, M. (2000) Intraspecific variation in Cannabis sativa L. based on intergenic spacer region of chloroplast DNA. Biological and Pharmceutical Bulletin. 23:727-730.
Kress, W. J.; Wurdack, K. J.; Zimmer, E. A.; Weigt, L. A.; Janzen, D. H. (2005) Use of DNA barcodes to identify flowering plants. Proceedings of the National Academy of Sciences. 102(23):8369-8374.
Kumar, S.; Tamura, K.; Jakobsen, I. B.; Nei, M. (2001) MEGA2: Molecular Evolutionary Genetics Analysis software. Bioinformatics. 17:1244-1255.
Neinhuis C.; Wanke, S.; Hilu K. W.; Muller, K.; Borsch, T. (2006) Phylogeny of Aristolochiaceae based on parsimony, likelihood, and Bayesian analyses of trnL-trnF sequences. Plant Systematics and Evolution 250:7-26.
Quandt, D.; Mueller, K.; Stech, M.; Hilu, K. W.; Frey, W.; Frahm, J. P.; Borsch, T. (2004) Molecular evolution of the chloroplast trnL-F region in land plants. In. Goffinet, B.; Hollowell, V.; Magill, R. (Eds.) Monographs in Systematic Botany: Molecular Systematics of Bryophytes: 13-37. Missouri Botanical Garden Press, St. Louis, Mo.
Sin, J.; Chan, C. (2004) Review of adverse events related to Chinese medicines in Hong Kong, January 2000-June 2004. Public Health and Epidemiology Bulletin, 13 (4): 60-66.
Wang, F.; Li, D. Z.; Yang, J. B. (2002) Molecular phylogeny of the Lardizabalaceae based on trnL-F sequences and combined chloroplast data. Acta Botanica Sinica. 44 (8): 971-977.
U.S. Food and Drug Administration (2001) Consumer advisory—FDA warns consumers to discontinue use of botanical products that contain aristolochic acid
U.S. Food and Drug Administration (2001) List of botanical ingredients of concern—FDA concerned about botanical products, including dietary supplements, containing aristolochic acid
U.S. Food and Drug Administration (2001) Letter to health professionals regarding safety concerns related to the use of botanical products containing aristolochic acid
U.S. Food and Drug Administration (2001) Letter to industry associations regarding safety concerns related to the use of botanical products containing aristolochic acid
Department of Health of Hong Kong (2004) Press release of Department of Health (8 Jun. 2004): updated control measures for medicinal herbs announced
Department of Health of Hong Kong (2004) Press release of Department of Health (24 Apr. 2004): Import and sale of specific Chinese herbs prohibited
Department of Health of Hong Kong (2004) Press release of Department of Health (13 Mar. 2004): Calls for suspension of the use of three Chinese herbs
Department of Health of Hong Kong (2004) Ban on import and sale of aristolochic acid (AA) containing herbs and products

We claim:

1. A method for determining whether a plant material is of the Aristolochia genus, comprising the steps of:
(a) obtaining a sample of DNA from an unauthenticated plant material, said DNA comprising a segment of chloroplast DNA;
(b) determining DNA sequence of said segment; and
(c) determining whether said DNA sequence comprises a fragment which is characteristic of the Aristolochia genus to assess a possibility that said plant material belongs to a species of the Aristolochia genus;
wherein said fragment is located within a trnL-trnF region or a psbA-trnH region of chloroplast DNA.

2. The method according to claim 1, wherein said step (c) is carried out by comparing said DNA sequence with a reference DNA sequence that encompasses said fragment.

3. The method according to claim 2, wherein said reference DNA sequence is, or encompasses, or is part of, a trnL-trnF region or a psbA-trnH region of chloroplast DNA.

4. The method according to claim 2, wherein said unauthenticated plant material is purported to be derived from a plant selected from the group consisting of Muxiang, Mutong, Fangji, Baiying and Madouling.

5. The method according to claim 2, wherein said fragment comprises a sequence of a genetic marker selected from the group consisting of Genetic Marker 1 to Genetic Marker 39.

6. The method according to claim 4, wherein said fragment comprises a sequence of a genetic marker selected from the group consisting of Genetic Marker 1 to Genetic Marker 39.

7. The method according to claim 2, wherein said reference DNA sequence is, or encompasses, or is part of, a sequence selected from the group consisting of SEQ ID NO: 1 to SEQ ID NO: 40.

8. The method according to claim 7, wherein said reference DNA sequence comprises a sequence of a genetic marker selected from the group consisting of Genetic Marker 1 to Genetic Marker 39.

9. The method according to claim 2, wherein said reference DNA sequence is a sequence selected from the group consisting of SEQ ID NO: 1 to SEQ ID NO: 40.

10. The method according to claim 7, wherein said reference DNA sequence is part of a sequence selected from the group consisting of SEQ ID NO: 1 to SEQ ID NO: 40, wherein said part is at least 80% by weight of said selected sequence.

11. The method according to claim 1, wherein said fragment is located within a trnL-trnF region.

12. The method according to claim 1, wherein said fragment is located within a psbA-trnH.

13. The method according to claim 3, said reference DNA sequence is, or encompasses, or is part of, a trnL-trnF region of chloroplast DNA.

14. The method according to claim 3, said reference DNA sequence is, or encompasses, or is part of, a psbA-trnH region of chloroplast DNA.

15. A genetic sequence, which is, or encompasses, or is part of, a sequence selected from the group consisting of SEQ ID NO: 1 to SEQ ID NO: 40, and is used for carrying out the method of claim 1.

16. The genetic sequence of claim 15, which is part of a sequence selected from the group consisting of SEQ ID NO: 1 to SEQ ID NO: 40, wherein said part is at least 80% by weight of said selected sequence.


SEQUENCE LISTING (73 sequences)

SEQ ID NO: 1 - DNA, 1031 bp, Aristolochia debilis
cgaaatcggtagacgctacggacttgattggattgagccttggtatggaaacctactaagtggtaacttccaaattcagagaaaccctggaattaaaaatgggcaatcctgagccaaatcctgttttgaaaaaaaaaaaaaaagttcagaataaagaataaaaaaaaggataggtgcagagactcaacggaagctgttctaacaaataaatggggttgactttactttactacataggtggaagaatccttctatcaaaactccagagtgggatgaccctagatctatatatatctatatatgtacgtatatgtactgaaatatcaaagattaatcacgacccgaatctatatatatatatttttctctatggtaaaaatttcagaagcagaaggaagaatcaaatagtcagtgatcaaatcgactatcacaccagagtttgatagatctttttaaagattgattaatcgggcgagacgagaataaagatagagtcccattctacatgtcaatactgacaacaatgaaatttctagtaagaggaaaatccgtcgactttagaaatcgtgagggttcaagtccctctatccccaataaaataaaaggcctattttactccctaagcatttatcctctttttttttttcatcagtggttcaaaattagatatgtttttcacttactctactctttcacaaatggatccgacctgaaatctttctctcttctcactgtgatagatattatatacgtacaaatgcccatccatatatggtatggatgggcaaagaatctctattacggaacccttcgcagttcatatcattactcttacacttccatttacaaagtctcccttttgaagatccaagaaattaccgactttatttaggtaagttaatgctttttgagtacctttcattgacatagacccaagccttttcttttagtaggatcatgcatcgagaatggtcgggatagctcagctggtagagcagaggactgaaaatcctcgtgtcaccagttcaaat

SEQ ID NO: 2 - DNA, 1080 bp, Aristolochia contorta
cgaaatcggtagacgctacggacttgattggattgagccttggtatggaaacctactaagtggtaacttccaaattcagagaaaccctggaattaaaaatgggcaatcctgagccaaatcctgttttgagaaaaaaaaaaaagagttcagaataaaaaaaaaataaaaaaaaaaaaaaggataggtgcagagactcaacggaagctgttctaacaaatcaaataaatggggttgactttacttttctacattggtagaagaatccttctatcaaaactcccgagtgggatgaccctatatctatatatgtacgtatatgtactgaaaatttatatatgtacgtatatgtactgaaatatcaaagattaatcacgacccgaatctatatatatatttttctctatgataaaaatttcagaagcagaaggaagaatcaaatagtcagtgatcaaatcgactatcacaccagagtttgatagatctttttaaagattgattaatcgggcgagacgagaataaagatagagtcccattctacatgtcaatactgacaacaatgaaatttctagtaagaggaaaatccgtcgactttagaaatcgtgagggttcaagtccctctatccccaataaaataaaaatccccaataaaataaaaggcctattttactccctaagcatttatcctctttttttttcatcagtggttcaaaattagattagatatgtttttcacttactctactctttcacaaatggatccaacctgaaatctttctctcttctcactgtgatagatatgatctacgtacaaatgcccatccatatatggatgggcaaagaatctctattacggaacccttcgcagttcatatcattactcttacacttccatttacaaagtctcccttttgaagatccaagaaattaccgactttatttaggtaagttaatgctttttgagtacttttcattgacatagacccaagccttttcttttagtaggatcatgcatcgagaatggtcgggatagctcagctggtagagcagaggactgaaaatcctcgtgtcaccagttcaaat

SEQ ID NO: 3 - DNA, 986 bp, Aristolochia fangchi
cgaaatcggtagacgctacggacttgattggattgagccttggtatggaaacctactaagtggtaacttccaaattcagagagaccctggaattaaaaatgggcaatcctgagccaaatcctgttttcagaaaaaaaaaggataggtgcagagactcaacggaagctgttctaacgaatggggttgactttactacattggtagaagaatccttctatcgaaactccagagtgggatgaccctagatatgtacgtatacgtactgaaatatcaaagattaatcacgacccgaatccatatttttttttttctaaaaatttcagaagcagaaggaagaatcgaatagtcagtgatcaaatcgactatcacaccagagtctgatagatctttttaaggattgattaatcgagtgagacgagaataaagatagagtcccattctacatgtcaatactgacaacaatgaaatttatagtaagaggaaaatccgtcgactttagaaatcgtgagggttcaagtccctctatccccaataaaataaaaatccccaataaaataaaaggcctattttactccctaagcatttatcctcttttttttttcatctgtggttcaaaattagattagatatgtttttcacttactctactctttcacaaaaggatccaacctgaaatctttctctcttctcactgtgatagatatgatctacgtacaaatgcccatccatatatggatgggcaaagaatctctattacggaacccttcgcagttcatatcattactcttacacttccatttacaaagtctcccttttgaagatacaagaaattaacgactttatttaggtaagttaatgctttttgagtacttttcattgacatagacccaagccttttcttttagtaggatcatgcatcgagaatggtcgggatagctcagctggtagagcagaggactgaaaatcctcgtgtcaccagttcaaat

SEQ ID NO: 4 - DNA, 957 bp, Aristolochia kaempferi
cgaaatcggtagacgctacggacttgattggattgagccttggtatggaaacctactaagtggtaacttccaaattcagagaaaccctggaattgaaaatgggcaatcctgagccaaatcctgttttcagaaaaaaaaaaggataggtgcagagactcaacggaagctgttctaacgaatggggttgactttactacattggtagaagaatccttctatcgaaactccagagtgggatgaccctacgtatgtacgtatacgtactgaaatatcaaagattaatcacgacccgaatccatatttttttttctaaaaatttcagaagcagaaggaagaatcgaatagtcagtgatcaaatcgactatcacaccagagtctgatagatctttttaaggattgattaatcgagtgagacgagaataaagatagagtcccattctacatgtcaatactgacaacaatgaaatttatagtaagaggaaaatccgtcgactttagaaatcgtgagggttcaagtccctctatccccaataaaaggcctattttactccctaagcatttatcctctttttttttttatccgtggttcaaaattagatatgtttttcacttactctactctttcacaaatggatccggccagaaatctttctctcttctcactgtgatagatatgatatacgtacaaatgcccatccatatatggatgggcaaagaatctccattacggaacctttcacagttcatatcattactcttacacttacatttacaaagcctcctttttgaagatccaacaaattaccgactttatttaggtaagttaatgctttttaagtccctttcattgacatagacccaagctttttcttttagtaggatcatgcatcgagaatggtcgggatagctcagctggtagagctgaggactgaaaatcctcgtgtcaccagttcaaat

SEQ ID NO: 5 - DNA, 984 bp, Aristolochia manshuriensis
cgaaatcggtagacgctacggacttgattggattgagccttggtatggaaacctactaagtggtaacttccaaattcagagaaaccctggaattaaaaatgggcaatcctgagccaaatcctgttttcagaaaaaaaaaaggataggtgcagagactcaacggaagctgttctaacgtctaacgagaagctgttctaacgaatggggttgactttactacattggtagaagaatccttctatcgaaactccagagtgggatgaccctagatatgtacgtatacgtactgaaatatcaaagattaatcatgacccgaatccatatttttttttctatgataaaaatttcagaagcagaaggaagaatcgaatagtcagtgatcaaatcgactatcacaccagagtctgatagatctttttaaggattgattaatcgagtgagacgagaaaaaagatagagtcccattctacatgtcaatactgacaacaatgaaatttatagtaagaggaaaatccgtcgactttagaaatcgtgagggttcaagtccctctatccccaataaaaggcctattttactccctaagcatttatcctcttttttttttatcagtggttcaaaattagatatgtttttcacttactctactctttcacaaatggatccggccagaaatctttctctcttatcactgtgatagatatgatatacgtacaaatgcccatccatatatggatgggcaaagaatcctcattacggaacctttcacagttcatatcattactcttacacttacatttacaaagtctcctttttgaagatccaacaaattaccgactttatttaggtaagttaatgctttttgagtccctttcattgacatagacccaagctttttcttttagtaggatcatgcatcgagaatggtcgggatagctcagctggtagagcagaggactgaaaatcctcgtgtcaccagttcaaat

SEQ ID NO: 6 - DNA, 956 bp, Aristolochia mollissima
cgaaatcggtagacgctacggacttgattggattgagccttggtatggaaacctactaagtggtaacttccaaattcagagaaaccctggaattaaaaatgggcaatcctgagccaaatcctgttttcagaaaaaaaaaaggataggtgcagaggctcaacggaagctgttctaacgaatggggttgactttactacattggtagaagaatccttctatcgaaactccagagtgggatgaccctagatatgtacgtatacgtactgaaatatcaaagattaatcacgacccgaatccatatttttttttctaaaaatttcagaagcagaaggaagaatcgaatagtcagtgatcaaatcgactatcacaccagagtctgatagatctttttaaggattgattaatcgagtgagacgagaataaagatagagtcccattctacatgtcaatactgacaacaatgaaatttatagtaagaggaaaatccgtcgactttagaaatcgtgagggttcaagtccctctatccccaataaaaggcctattttactccctaagcatttatcctcttttttttttatcagtggttcaaaattagatatgtttttcacttactctactctttcacaaatggatccggccagaaatctttctctcttctcactgtgatagatatgatatacgtacaaatgcccatccatatatggatgggcaaagaatctccattacggaacctttcacagttcatatcattactcttacacttacatttacaaagtctcctttttgaagatccaacaaattaccgactttatttaggtaagttaaagctttttaagtccctttcattgacatagacccaagctttttcttttagtaggatcatgcatcgagaatggtcgggatagctcagctggtagagcagaggactgaaaatcctcgtgtcaccagttcaaat

SEQ ID NO: 7 - DNA, 964 bp, Aucklandia lappa
cgaaatcggtagacgctacggacttaattggattgagccttggtatggaaacttactaagtgataactttcaaattcagagaaaccctggaattaataaaaatgggcaatcctgagccaaatcacgttttccgaaaacaaacaaaggttcagaaagcgaaaataaaaaaggataggtgcagagactcgatggaagctgttctaacgaatggagttgattgtcttacgttggtagaggaatccttctatcgaaacttcataaaagatgaaggataaacctgtatacataatatagaagaattgttgtgaatcgattccgaaagaatcgaatattcattgatcaaacaattcactccataatctgatagatcttttgaagaacttattaatcggacgagaataaagatagagtcccgttctacatgtcaataccggcaacaatgaaatttatagtaagaggaaaatccgtcgatttaaaaaatcgtgagggttcaagtccctctatccccaataaaataaaaatccccaataaaataaaaggcctattttactccctaagcatttatcctcttttttttttcatcagtggttcaaaattagattagatatgtttttcacttactctactctttcacaaaaggatccaacctgaaatctttctctcttctcactgtgatagatatgatctacgtacaaatgcccatccatatatggatgggcaaagaatctctattacggaacccttcgcagttcatatcattactcttacacttccatttacaaagtctcccttttgaagatacaagaaattaacgactttatttaggtaagttaatgctttttgagtactttttattgacatagacccaagccttttcttttagtaggatcatgcatcgagaatggtcgggatagctcagctggtagagcagaggactgaaaatcctcgtgtcaccagttcaaat

SEQ ID NO: 8 - DNA, 963 bp, Vladimiria souliei var. mirabilis
cgaaatcggtagacgctacggacttaattggattgagccttggtatggaaacttactaagtgataactttcaaattcagagaaaccctggaattaataaaaatgggcaatcctgagccaaatcacgttttccgaaaacaaacaaaggttcagaaagcgaaaatcaaaaaggataggtgcagagactcgatggaagctgttctaacgaatggagttgattgtcttacgttggtagaggaatccttctatcgaaacttcataaaagatgaaggataaacctgtatacataatatagaagaattgttgtgaatcgattccatattgaagaaagaatcgaatattcattgatcaaacaattcactccataatctgatagatcttttgaagaacttattaatcggacgagaataaagatagagtcccgttctacatgtcaataccggcaacaatgaaatttatagtaagaggaaaatccgtcgatttaaaaaattgtgagggttcaagtccctctatccccaaaaataccatttgactccctaattatttctcgtatcctttttatttattttatccttttttccttagcggttcaaaattccttatcttttctttctcattcactactctttatacatcattctctgctctttatacaaatggatctgagcggaaatgctgttctcttatcacatgtgatatatatgatacatgtacaaatgaacatctttgagcaaggaatccccatttgaatgattcacgatcgatatttttattcatactgaaacttacaaagttgttcttttgacaaattataggacctggatgaggctttgtaataccctttcaattgacatagacccaagttatctagtaaaatgaaagtgaggatgagacatcaggaatagtcgggatagctcagttggtagagcagaggactgaaaatcctcgtgtcaccagttcaaat

SEQ ID NO: 9 - DNA, 1010 bp, Akebia quinata
cgaaatcggtagacgctacggacttgaatggattgagccttggtatggaaacctactaggtgataactttcaaattcagagaaaccctggaatgaaaaatgggcaatcctgagccaaatcctgttttcagaaaaaaaaaggttcagaaagcgagattaaaaaaataaaggaaggataggtgcagagactcaatggaagctgttctaatgaatggagttggctgcgttgcgttcgtagaggaatccttctattgaaactccagaaaggatgaaggatgaacctatatacgtacgtatacgtactgaaatatcaaaagattaatgacgatcaaaatattttttttatatgaaaaacggaagaattattgtgaatcaatttcaagttgaaggaaaaatcgaatattcactgatcaaatcagtcattccatagtctgatagatcttttgaagaactgactaatcggacgagaataaagatagagtcccattctacatgtcaataccgacaataatgaaatttatagtaagaggaaaatccgtcgactttagaaatcgtgagggttcaagtccctctatccccaagaaaaaggcccgttttactccttaactatactatgttatcctttttttgtcagcggttccaaattcgctaagtttctcattcactccactctttcacaaatggatcggagcataaatgtttctctcttatcacaagtcaagtcttgtgatatatacgtacaaatgcacacctatgagcaaggaatccccatttgaatcatttaatttacagtccatatgatttacttaaacttacaaagttacaaagtcttctttttgaagatgcaagaaattcccgggcctgggtacgactttgtaatgcttttttgaatctctttaattgacataaactcaagccctctagtggtatagagatgatgcatcgggaaaggtcgggatagctcagctggtagagcagaggactgaaaatcctcgtgtcaccagttcaaat

SEQ ID NO: 10 - DNA, 1010 bp, Akebia trifoliata
cgaaatcggtagacgctacggacttgattggattgagccttggtatggaaacctactaagtgataactttcaaattcagagaaaccctggaatgaaaaatgggcaatcctgagccaaatcctgttttcagaaaaaaaaaggttcagaaagcgagattaaaaaaataaaggatggataggtgcagagactcaatggaagctgttctaacgaatggagttggctgcgttgcgttcgtagaggaatccttctattgaaactccagaaaggatgaaggatgaacctatatacgtacgtatacgtactgaaatatcaaaagattaatgacgatcaaaatattttttttatatgaaaaacggaagaattattgtgaatcaatttcaagctgaaggaaaaatcgaatattcactgatcaaatcagtcattccatagtctgatagatcttttgaagaactgactaatcggacgagaataaagatagagtcccattctacatgtcaataccgacaataatgaaatttatagtaagaggaaaatccgtcgactttagaaatcgtgagggttcaagtccctctatccccaagaaaaaggcccgttttactccttaactatactatgttatcctttttttgtcagcggttccaaattcgctaagtttctcattcactccactctttcacaaatggatcggagcataaatgtttctctcttatcacaagtcaagtcttgtgatatatacgtacaaatgcacacctatgagcaaggaatccccatttgaatcatttaatttacagtccatatgatttacttaaacttacaaagttacaaagtcttctttttgaagatgcaagaaattcccgggcctgggtacgactttgtaatgcttttttgaatctctttaattgacataaactcaagccctctagtagtatagagatgatgcatcgggaaaggtcgggatagctcagctggtagagcagaggactgaaaatcctcgtgtcaccagttcaaat

SEQ ID NO: 11 - DNA, 1009 bp, Akebia trifoliata var. australis
cgaaatcggtagacgctacggacttgattggattgagccttggtatggaaacctactaagtgataactttcaaattcagagaaaccctggaatgaaaaatgggcaatcctgagccaaatcctgttttcagaaaaaaaaaggttcagaaagcgagattaaaaaaataaaggaaggataggtgcagagactcaatggaagctgttctaacgaatggagttggctgcgttgcgttcgtagaggaatccttctattgaaactccagaaaggatgaaggatgaacctatatacgtacgtatacgtactgaaatatcaaaagattaatgacgatcaaaatattcttttatatgaaaaacggaagaattattgtgaatcaatttcaagttgaaggaaaaatcgaatattcactgatcaaatcagtcattccatagtctgatagatcttttgaagaactgactaatcggacgagaataaagatagagtcccattctacatgtcaataccgacaataatgaaatttatagtaagaggaaaatccgtcgactttggaaatcgtgagggttcaagtccctctatccccaagaaaaaggcccgttttactccttaactatactatgttatcctttttttgtcagcggttccaaattcgctaagtttctcattcactccactctttcacaaatggatcggagcataaatgtttctctcttatcacaagtcaagtcttgtgatatatacgtacaaatgcacacctatgagcaaggaatccccatttgaatcatttaatttacagtccatatgatttacttaaacttacaaagttacaaagtcttctttttgatgatgcaagaaattcccgggcctgggtacgactttgtaatgcttttttgaatctctttaattgacataaactcaagccctctagtagtatagagatgatgcatcgggaaaggtcgggatagctcagctggtagagcagaggactgaaaatcctcgtgtcaccagttcaaat

SEQ ID NO: 12 - DNA, 779 bp, Clematis armandii
cgaaatcggtagacgctacggacttggttggattgcagccttgcatatggcaaacatactaaggtgataactttcaaattcagagaaaccccggaataaaaaatgggcaatcctgagccaaatccttttttcagaaaacaaaagggcttcagaaagcaagaataaaaaaaaaaggataggtgcagagactaaatggaagctgttctaacgaatagagttgactgcgttgatcgaggattccttctatttaaactacagaaaggatgaacccgtatacatataaaaatataaaagattaatgacaatcaaaatatttttttcttatatgatatgcaaaatgtaagaattcttgtgatgtgaattcattcaaagtttaagtaagaatcgaatattcactgatcaaataagtcactctataatctgatagatcttttaaagaactaattggacgagaataaagatagagtcccattatacatgtcaacatcaatactgacaaaaatgaaatttatagtaagaggaaaatccgtcgactttttaaatcgtgagggttcaagtccctctatccccaaaaaaaagtttactgtaccccccaactatttagttaattaattcttttttttttcttttttctaagactttttaatgcttttttagtctatttaattgatatacccaacaagcactctaaatagagtggggatgccgcatcgagaagagtcgggatagctcagttggtagagcagaggactgaaaatcctcgtgtcaccagttcaaat

SEQ ID NO: 13 - DNA, 776 bp, Clematis montana
cgaaatcggtagacgctacggacttggttggattgagccttgatatggaaacatactaagtgataactttcaaattcagagaaaccccggaataaaaaatgggcaatcctgagccaaatccttttttcagaaaacaaaagggcttcagaaagcaagaataaaaaaaaaggataggtgcagagactaaatggaagctgttctaacgaatagagttgactacgttgatcgaggattccttctatttaaactacagaaaggatgaacccgtatacatataaaaatataaaagattaatgacaatcaaaatatttttttcttatatgatatgcaaaatgtaagaattcttgtgatgtgaatcgattcaaagtttaagtaagaatcaaatattcactgatcaaataagtcactctataatctgatagatcttttaaagaactaattggacgagaataaagatagagtcccattatacatgtcaacatcaatactgacaaaaatgaaatttatagtaagaggaaaatccgtcgactttttaaatcgtgagggttcaagtccctctatccccaaaaaaaagtttactgtaccccccaactatttagttaattaattcttttttttttttcttttttctaagactttttaatgcttttttagtctatttaattgatatacccaacaagcactctaaatagagtggggatgccgcatcgggaagagtcgggatagctcagttggtagagcagaggactgaaaatcctcgtgtcaccagttcaaat

SEQ ID NO: 14 - DNA, 1034 bp, Cocculus orbiculatus
cgaaatcggtagacgctacggacttgattggattgagccttggtatggaaacctactaagtgaaaactttcaaattcagagaaaccctggaattaaaaatgggcaatcctgagccaaatcctgttttcagaaaacaaaaaagagttcagaaagcgagactcaaaaatggataggtgcagagactcaatggaagctgttctaacaaatggagttgattgcgttgcgttagtagagtaatccttctattgaaactccagaaaggatgaacttatctatatacatacgtctacgtactgaaatatcaaagattcatgatgatccgaatctgtatttatgaaaaatggaagaatttttgtgaatcaactccaagttaaaggaagagtcgaacattcactgatcaaatcagtcactccatagtctgatagatcttttgaagaactgactaatcggacgagaataaagatagagtcccattctacatgtcaataccgacaacaatgaaatttatagtaagaggaaaatccgtcgactttagaaatcgtgagggttcaagtccctctatccccaataaaaataaaaggcccactttactccctaattagttatccttgtattctttcttttcggcagcaattcaaaattagctatgtttctcgttcactctactctttcacaaatggatcggaagggaagtgtttctctcttatcacaagtattgtgatatatacactatacgtacaaatgagcatctatgggcaaggaatcccaatttgaatcattcacagtccgtatcattattttaaacgtcaaaagtattctgtttgaagatccaagaaattccaaggactgggtaaaactttgttttgtaatgcttttttagcctctttattttttagtctctttaatcgacataggcccaagaattgacataggcccaagccctctagtagtatggggatgatgcatcgagaggggccgggatagctcagttggtagagcagaggactgaaaatcctcgtgtcaccagttcaaat

SEQ ID NO: 15 - DNA, 1022 bp, Stephania tetrandra
cgaaatcggtagacgctacggacttgattggattgagccttggtatggatacctactaagtgaaaactttcaaattcagagaacccctggaattaaaaatgggcaatcctgagccaaatcctgttttcagaaaacaaaaaagggtttagaataggagactcaaaaatggataggtgcagagactcaatggaagctgttctaagaaatggagttgattgcgttgcgttagtggaggaatccttctattgaaactccagtaaaggatgaacttatctatatacatacgtctacgtactgaaatatcaaagattcatgatgatccgaatctgtatttatgaaaaatggaagaatttgtgtttttgtaaatcaattccaagttgaaggaagaatcgaacattcactgatcaaatcagtcactccatagtctgataggtcttttgaagaactgactaatcggacgagaataaagatagagtcccattctacgtgtcaataccgacaacaatgaaatttatagtaagaggaaaatccgtcgactttagaaatcgtgagggttcaagtccctctatccccaagaaaaataaaaggccaattttactccctaacgagttatctttttttttttttttctttcttttcggcagcagttccaaattcgctatgtttctcgttcactctactctttcccaaaccaaatggatgggaagagaaatgtttctcttttatcacaatacttgtgatatatacgtacaaatgagcatctatgggcaaggaatcccaatttgaatcattcacagtccgtattatattctaattttgaacgccaaaagtattctgtttgatgaagatccaagaaattccaagaactgggtaagattttgttttgcaatgcttttttagtctctttaattgacataggtccaagccctctagcagtatggggatgacgcatcgagagaggggccgggatagctcagttggtagagcagaggactgaaaatactcgtgtcaccagttcaaat

SEQ ID NO: 16 - DNA, 1043 bp, Solanum dulcamara
cgaaatcggtagacgctacggacttaattggattgagccttggtatggaaacttactaagtgatcactttcaaattcagagaaaccctggaattaacaaaaatgggcaatcctgagccaaatcctgttttctgaaaacaaacaaaggttcagaaaaaaaggataggtgcagagactcaatggaagctattctaacaaatggaattaaatgcgttggtagaggactctttacatcgaaacttcagaaagaaaaagaatgaagtgaaggataaacgtatatacatacgtattgaatactatatcaaatgattaatgacgacccgaatccgtagtttttctataaaaaatcgaagaattggtgtgaatccattctacattgaagaaagaatcgaatattcattgatcaaatcattcactccatagtctgatagatcttttgaagaactgattaatcggacgagaataaagatagagtcccgttctacatgtcaataccggcaacaatgaaatttctagtaaaaggaaaatccgtcgactttaaaaatcgtgagggttcaagtccctctatccccaaaaagactatttaactccccaactatttatccgaccccctttccttagcggttgcaaattccttatctttatcattcactctattcttttagaaatggattttttttctaagcgtaaatggttttctcttataacaagcctttatgatatctatgatacacataaaaatgaacatctttgagcaaggaatccctagttgaatgattcccgatcaatacaatatcattactcatactgaaacttacaaagtcatctttttgaagatcgaagaaattacccggtttgagaaaattttttaatctactttgtgtccttttaattgacatagcccccagttctatgataaaatgaggataccacgcaaaggactaaaaatcctcaggttagcggttccaatcgagattgggaatagccgggatagctcagttggtagagcagaggactgaaaatcctcgtgtcaccagttcaaat

SEQ ID NO: 17 - DNA, 1041 bp, Solanum lyratum
cgaaatcggtagacgctacggacttaattggattgagccttggtatggaaacttactaagtgatcactttcaaattcagagaaaccctggaattaacaaaaatgggcaatcctgagccaaatcctgttttctgaaaacaaacaaaggttcagaaaaaaaggataggtgcagagactcaatggaagctattctaacaaatggagttaaatgcgttggtagaggactctttacatcaaaacttcagaaagaaaaagaatgaagtgaaggataaacgtatatacatacgtattgaatactatatcaaatgattaatgacgacccgaatccgtagtttttctataaaaaatcgaagaattggtgtgaatccattctacattgaagaaagaatcgaatattcattgatcaaatcattcactccatagtctgatagatcttttgaagaactgattaatcggacgagaataaagatagagtcccgttctacatgtcaataccggcaacaatgaaatttatagtaaaaggaaaatccgtcgactttaaaaatcgtgagggttcaagtccctctatccccaaaaagactatttaactccccaactatttatccgaccccctttccttagcggttccaaattccttatctttctcattcactctattcttttagaaatggatttttttctaagcgtaaatggttttctcttatcacaagcctttatgatatctatgatacacatagaaatgaacatctttgagcaaggaatccctagttgaaagattcccgatcaatacaatatcattacttatactgaaacttacaaagtcatctttttgaagatcgaagaaattccccggtttgagaaaattttttaatctattttagtccttttaattgacatagaccccagttctatgataaaatgaggataccatgcaaaggactaaaaatcctcaggttagcggttccaatcgagattgggaatagccgggatagctcagttggtagagcagaggactgaaaatcctcgtgtcaccagttcaaat

SEQ ID NO: 18 - DNA, 847 bp, Cardiocrinum giganteum
cgaaatcggtagacgctacggacttgattaaattgagccttggtatggaaacctgctaagtgataacctccaaattcagagaaaccccggaattaaaaatgggcaatcctgagccaaatctttattttgagaaaaagggtttaatttatagtttttataatataaaactacaataaaaaaagataggtgcagagactcaatggaagctgttctaacgaatgaagttgattacgttgcgctggtagccggaatccttctatcaaaattacggagaggatgcccgccctatatacgtatatatacatactgacatagtaaacgattgattaatcacggcccgaatccattatataatatatatatatatgaaaaatttaagagttattgtaaatccattccatattccaatcaaagtttaaggaagaatcaaatattcagtgatcaaattattcgttccagagtctgtatagatctttttaaaaactgattattcggacgagaataaagagagagtcccattttacgtgtcaataccgacaacaatgaaatttatagtaaaaggaaaatccgtcgactttataagtcgtgagggttcaagtccctctatccccaataaaagcccattttaagtactaacttaactatacttcctaactactttttatcttatctttttttaatctaagaaattcaggggctgggtaagattttttagtactttcttaattttttagtctctttaattgacataggtaaagtgctctactaggataatgcgcgagaaaaggtcgggatagctcagttggtagagcagaggactgaaaatcctcgtgtcaccagttcaaat

SEQ ID NO: 19 - DNA, 847 bp, Cardiocrinum yunanense
cgaaatcggtagacgctacggacttgattaaattgagccttggtatggaaacctgctaagtgataacttccaaattcagagaaaccccggaattaaaaatgggcaatcctgagccaaatctttattttgagaaaaagggtttaatttatagtttttataatataaaactacaataaaaaaagataggtgcagagactcaatggaagctgttctaacgaatgaagttgattacgttgcgctggtagccggaatccttctatcaaaattacggagaggatgcccgccctatatacgtatatatacatactgacatagtaaacgattgattaatcgcggcccgaatccattatataatatatatatatatgaaaaatttaagagttattgtaaatccattccatattccaatcaaagtttaaggaagaatcaaatattcagtgatcaaattattcattccagagtctgtatagatctttttaaaaactgattattcggacgagaataaagagagagtcccattctacgtgtcaataccgacaacaatgaaatttatagtaaaaggaaaatccgtcgactttataagtcgtgagggttcaagtccctctatccccaataaaagcccattttaagtactaacttaactatacttcctaactactttttatcttatctttttttaatctaagaaattcaggggctgggtaagattttttaatactttcttaattttttagtctctttaattgacatagataaagtgctctactaggataatgcgcgagaaaaggtcgggatagctcagttggtagagcagaggactgaaaatcctcgtgtcaccagttcaaat

SEQ ID NO: 20 - DNA, 819 bp, Dioscorea cirrhosa
cgaaatcggtagacgctacggacttgattgaattgagccttggtatggaaacctgctaagtgataacttccaaattcagagaaaccctggaatttgaaatgggcaatcctgagccaaatctttatttgttttgttttttataaaccctttggtttataaaaaaacaaaacaaactagaatcaaaaatcaaaaaggataggtgcagagactcaatggaagctgttctaacgaatggaattgactacgttatgttggtagttggaaaacctacattgaaattatgaaaaggggagttctatatacccaatacgtacgtatacatactaacatattaaaggattaatcacgacccgaatccattattttatttatatatattatgaaaaaatggaaagttatattgtgaatctatttcaaccgaagttgaaggaagaatcaaatatttaatgatcaaatcattgattctagagtctgatagatcttttgaaaaacgaattaatcggacgagaataaagagagagtcctgctctacatgtcaatactgacaacaatgaaatttatagtaagaagaaaatccgtcgactttagaaatcgtgagggttcaagtccctctatccccaaaaaagtccattttatttcctaattatttatactcttttttttgaagatcgaaggactggataagattttttaagagtccccttaattgagatagatagatataagtactctagcaggatgatgcgagaaaagaaaatggttgggatagctcagttggtagagcagaggactgaaaatcctcgtgtcaccagttcaaat

SEQ ID NO: 21 - DNA, 343 bp, Aristolochia contorta
ggtatgcatgaacgtaatgctcacaacttccctctagacctggctgctcttgaagctccatctacaaatggataatacatcgttatttgtgtatacgagtcgttgaaggagcaatacccaatccttttgttcttatcaagaggtgggtattgttccgctcaattcactatcaactcattagagttttactcatattttactcatttttactcatatcgcatatatattatatgtatatgcattttatccatagtttaaagttaaaaaaaaaaaaaaaggaaaggccaggggcggatgtagccaagtggatcaaggcagtggatttgtgaatccaccatgcgcg

SEQ ID NO: 22 - DNA, 349 bp, Aristolochia debilis
ggtatgcatgaacgtaatgctcacaacttccctctagacctggctgctcttgaagctccatctacaaatggataatacatcgttattagtgtatacgagtcgttgaaggagcaatacccaatccttttgttcttatcaagaggtgggtattgttccgctcaattcactagagttttactcatattttactcatatcgcatatataatatattatatgctatgcattttatccatttttttagtttaaagttaaaaaaaaaaatgaaaaaggaaaggttaaaaaggaaaggccaggggcggatgtagccaagtggatcaaggcagtggatttgtgaatccaccatgcgcg

SEQ ID NO: 23 - DNA, 276 bp, Aristolochia fangchi
ggtatgcatgaacgtaatgctcacaacttccctctagacctggctgctcttgaagccccatccacaaatggataatacttcggtattagtgtatacgaatcgttgaaggagcaatacccaattcttgtttttatcaagaggtgggtattgttctgctcaattcactagtgttttactcattacagcggattactccttttttgaagttaaagaaagggcaggggcggatgtagccaagtggatcaaggcagtggatttgtgaatccaccatgcgcg

SEQ ID NO: 24 - DNA, 261 bp, Aristolochia kaempferi
ggtatgcatgaacgtaatgctcacaacttccctctagacctggctgctcttgaagctccatccacaaatggataatacttcggtattagtgtatacgaatcgttgaaggagcaatacccaattcttgtttttatcaagaggtgggtattgttctgctcaattcactagtgttttactcctttttttttaagttaaagaaagggcaggggcggatgtagccaagtggatcaaggcagtggatttgtgaatccaccatgcgcg

SEQ ID NO: 25 - DNA, 274 bp, Aristolochia manshuriensis
ggtatgcatgaacgtaatgctcacaacttccctctatacctggctgctcttgaagctccatccacaaatggataatacattcggtattagtgtatacgaatcgttgaaggagcaatacccaattcttgtttttatcaagaggcgggtattgttctgctcaattcactagtgttttactcgcggattactccttttttttttaagttaaagaaagggcaggggcggatgtagccaagtggatcaaggcagtggatttgtgaatccaccatgcgcg

SEQ ID NO: 26 - DNA, 260 bp, Aristolochia mollissima
ggtatgcatgaacgtaatgctcacaacttccctctagacctggctgctcttgaagctccatccacaaatggataatacttcggtattagtgtatacgaatcgttgaaggagcaatacccaattcttgtttttatcaagaggtgggtattgttctgctcaattcactagtgttttactccttttttttttagttaaagaaagggcaggggcggatgtagcaagtggatcaaggcagtggatttgtgaatccaccatgcgcg

SEQ ID NO: 27 - DNA, 517 bp, Aucklandia lappa
ggtatgcatgaacgtaatgctcataatttccctctagacttagctgctatcgaagctccatctacaaatggataagactttggtctgattgtataggagtttttgaactaaaaaaggagcaatagcttccctcttgttttatcaagagggcgttattgctcctttttttatttagtagtatttaccttacatagtttctttaaaaataacaagggggttttatagtttggttcgattagcatgttttatctttgtattaatttctattataggtttatatatccttttcccaatcgtttatgaagttttatttccaactcaatttcaatctaaaatagataaaaattataattttgattatttattgcttttatttcagaaataagaaagaaataatatgctcttttttttatgttaatggaaaaataaaatatagtaatagtagataatactagataataggggcggatgtagccaagtggatcaaggcagtggatttgtgaatccaccatgcgcg

SEQ ID NO: 28 - DNA, 518 bp, Vladimiria souliei var. mirabilis
ggtatgcatgaacgtaatgctcataatttccctctagacttagctgctatcgaagctccatctacaaatggataagactttggtctgattgtataggagtttttgaactaaaaaaggagcaatagcttccctcttgttttatcaagagggcgttattgctcctttttttatttagtagtatttaccttacatagtttctttaaaaataacaaggggggttttatagtttggttcgattagcgtgttttatctttgtattaatttctattataggtttatatatccttttcccaatcgtttatgaagttttatttccaactcaatttcaatataaaatagataaaaattataattttgattatttattgcttttatttcagaaataagaaagaaataatatgctcttttttttatgttaatggaaaaataaaatatagtaatagtagataatactagataataggggcggatgtagccaagtggatcaaggcagtggatttgtgaatccaccatgcgcg

SEQ ID NO: 29 - DNA, 617 bp, Akebia quinata
ggtatgcatgaacgtaatgctcacaacttccctctagacctagctgctgtcgaagctccagcgacaaatggataagatttccgtcttagtgtatccgagttattgaaggtgaaggagtaatacccaatttcttgttctatcaagaggacgggtattgttccttcatttagttttagtagtttttactcgcagaatctgctctttttttttttatttattttaactaaaataaaaatgaatataggtttattcattattgagtatcatacttttgttctgtataaaatatataatttatgaatttatatatgttatatgttcctcgaaattttttgacttgacttttttcataaattatggaaattatgaaactaaagctacttttttataaaggattggtcttgcatgcccaatatctgtatcttagtatatcttagtatcaagaaaagtaagaactactcaattaaaaaactcaattaaaaaatgaaaaataacaaaaaaagaaattcaatttctaccatatggtttttttttatttttttgtgagtttgagggagtaggggggcggatgtagccaagtggctcaaggcagtggatttgtgaatccaccatgcgcg

SEQ ID NO: 30 - DNA, 617 bp, Akebia trifoliata
ggtatgcatgaacgtaatgctcacaacttccctctagacctagctgctgtcgaagctccagcgacaaatggacaagatttccgtcttagtgtatccgagttattgaaggtgaaggagtaatacccaatttcttgttctatcaagaggacgggtattgttccttcatttagttttagtagtttttactcgcagaatctgctcttttttttttatttattttaactaaaataaaaatgaatataggtttattcattattgagtatcatacttttgttctgtataaaatatataatttatgaatttatatatgttatatgttcctcgaaattttttgacttgacttttttcataaattatggaaattatgaaactaaagctacttttttataaaggattggtcttgcatgcccaatatctgtatcttagtatatcttagtatcaagaaaagtaagaactactcaattaaaaaataaaaaatgaaaaataacaaaaaaagaaattcaatttctaccatatggttttttttttttttttatttttttgtgagtttgagggagtaggggggcggatgtagccaagtggctcaaggcagtggatttgtgaatccaccatgcgcg

SEQ ID NO: 31 - DNA, 608 bp, Akebia trifoliata var. australis
ggtatgcatgaacgtaatgctcacaacttccctctagacctagctgctgtcgaagctccagcgacaaatggataagatttccgtcttagtgtatccgagttattgaaggtgaaggagtaatacccaatttcttgttctatcaagaggacgggtattgttccttcatttagttttagtagtttttactcgcagaatctgctcttttttttttttatttattttaactaaaataaaaatgaatataggtttattcattattgagtatcatacttttgttctgtataaaatatataatttatgaatttatatatgttatatgttcctcgaaattttttgacttgacttttttcataaattatggaaattatgaaactaaagctacttttttataaaggattggtcttgcatgcccaatatctgtatcttagtatatcttagtatcaagaaaagtaagaactactcaattaaaaaatgaaaaataacaaaaaaagaaattcaatttctaccatatggttttttttttttatttttttgtgagtttgagggagtaggggggcggatgtagccaagtggctcaaggcagtggatttgtgaatccaccatgcgcg

SEQ ID NO: 32 - DNA, 456 bp, Clematis armandii
ggtatgcatgaacgtaatgctcacaactcccctctagatctagcggctgttgaagttccatctacaaatggctaagacttaggtcttagtgtatgctagtcttagtgtatatgagtcgttgaagttgcaggagtaataccccaattcttgctctgtcaagaggccgggcattgctcctgcgttttgttttaatagtgttttatttgcatttttcataatgattttttatttttttttgaagtcatttcatttttgaatttgaagtaaaaaaaaaaaatgactcgaatgggggattggttgttgaattcttgattatcatactctcgttctgtacgtatataaaccaatccaatataatatgttcctaaaaattttgaaacaatttatttgagaaagcggaggggcggatgtagccaagtggatcaaggcagtggatttgtgaatccaccatgcgcg

SEQ ID NO: 33 - DNA, 447 bp, Clematis montana
ggtatgcatgaacgtaatgctcacaacttccctctagatctagcggctgttgaagttccatctacaaatggctaagacttaggtcttagtgtaggctagtcttagtgtatatgagtcgttgaagttgcaggagcaatacctcaattcttgttctgtcaagaggccgggcattgctcctgcgttttgttttaatagtgttttatttgcattttgcataatgattttttcttttttttttgaagtaatttaatttgaagtaaaaaaaaaatgactcgaatggaggattggttggtgaattcttgattatcatactctcgttctgtacatatataaaccaatccaatataatatgttcctaaaaatttggaaacaatttatttgagaaagcggaggggcggatgtagccaagtggatcaaggcagtggatttgtgaatccaccatgcgcg

SEQ ID NO: 34 - DNA, 643 bp, Cocculus orbiculatus
ggtatgcatgaacgtaatgctcacaacttccctctagatctagcagctgttgaagctccatctacaaatggctaagatttcggtcttagtgtatacgagtcgttgaagttgaaagagcaatacccaattttttgttctgtcaagggggcgggtattgctccttcaattagggttagtattgttttgttttactcacataagcatttttttcattttctatttatttatttcaacttataaaattcagaaaaaagcattcaattaataaggatttttttgagtattttattatgccttgattctgcactaacgaatttgtaatttctatatgttatttaggtgataggtttcccgacatctttttcttcctaaagttcttttattattctttgattttttttcatattttatttctattagtttagttttaactaaactagaaaaataaaataaaagattcccttttaggtacaatatctttatttcagtatattctaaagagatgcgtaagttaggaaagacttaacttaaacatgtaaaagaaatgaatactcattcataatagaaattgaatgtttttgggggttaagggcggatgtggccaagtggattaaggcagtgatttgtgaatccaccatgcgcg

SEQ ID NO: 35 - DNA, 697 bp, Stephania tetrandra
ggtatgcatgaacgtaatgctcacaacttccctctagatctagctgctgttgaagatctatctacaaatggctaagatttcggtcttagtgtatatgagccgttgaatttgaaggagcaatacccaattttttgttctgtcaagggggcgggtattgctccttcggttggggttaggttagtattgttttgttttactcacataagtatttttccattttctatttctttatttcaacttacgaaattcagaaaaaaaaaaaagaattaatatggatttaattattgagtattatattatgctttgattctgtactaattaatttaaaatttctaaatgttattttcgtaatatgtttcccgacatcctttatattcctaaaaaatcsttatattcctaaaaaaaaagttctttttttgaatattttctttttctagttttctagtttagcaaaaaaattaatataaagaaagcaaaggattaccttttttttttattaagtacagtatccttacctcagtatattctaaagagatgcgtaagttaacaaagaaaagacttaattgaaacatgtaaaaaagccgaatgaaatactcattcataatcgaaattgaatctttttgggggtaaggtaaagtataagggcggatgtagccaagtggattaaggcaatggatttgtgaatccaccatgcgcg

SEQ ID NO: 36 - DNA, 574 bp, Solanum dulcamara
ggtatgcatgaacgtaatgctcataacttccctctagacctagctgctatcgaagctccatctacaaatggataagatccgagcctagtctataggaggttttgaaaagaaaggagcaataaccattttcttgttctatcaagagggtgctattgctcctttctttttttatttttctttattaatttcgtagtattttactgacatagacttttttgtttacattatcgaaaaagaaggagagggtatttgcctgcatttattcatgattgagtattcgagtttgattttatatttattgaaaattgtagaaatataacttgttcctcttgttgctaatgttactatatcttttttatttcatttcacaaaaaatataatttttacttcatattcttatctttgaaataataatcgaaataataatatcattgaaataagaaagaagagaaatattcgaactttattcttttgttttcgaatttaaataatgtaaaaatgtactgtaagtaggcgagggggcggatgtagccaagtggatcaaggcagtggatttgtgaatccaccatgcgcg

SEQ ID NO: 37 - DNA, 567 bp, Solanum lyratum
ggtatgcatgaacgtaatgctcataacttccctctagacctagctgctatcgaagctccatctacaaatggataagatccgagcctagtctataggaggttttgaaaagaaaggagcaataaccattttcttgttctatcaagagggtgctattgctcctttctttttttctttttctttattaatttcctagtattttactgacatagacttttttgtttacattatagaaaaaatagaaaaagaaggagagagtatttgagtttgattttatatttattgaaaattgtagaaatataacttgttcctcttgttgctaatgttactatatcttttttatttcatttcacaaaaaatataatttttacttcatattcttatctttgaaataataaatattcttatctttgaaataataatatcattgaaataagaaagaagagaaatattcgaactttactcttttgttttcgaatttaaataatgtaaaaatggactgtaagtagtcgagggggcggatgtagccaagtggatcaaggcagtggatttgtgaatccaccatgcgcg

SEQ ID NO: 38 - DNA, 489 bp, Cardiocrinum giganteum
ggtatgcatgaacgtaatgctcacaatttccctctagactagctgctgttgaagttccatctacaaatggataagacttccattctgtcttagtgtatacgaatcattgatggagcaatatctttgtatcttgctaaagatattgctccctttttttggccaatcattgtgggtataatggtagatgccctagaccaagtgactattatttctttctcctcccttatgtttagtttttcaatttttgccgataaatgattagctacaaaaggatttttttttattgaacgtgtcacaggggattactcctttattacattacatttttaaaattggcattctatgcccaatatatcgatctaagtatgaaggtaagaataaatacaataatgatgaatggaaaaagaaaaaagctaaaatcttttagctagataaggggcggatgtagccaagtgatcaggcagtggatttgtgaatccaccatgcgcg

SEQ ID NO: 39 - DNA, 492 bp, Cardiocrinum yunanense
ggtatgcatgaacgtaatgctcacaatttccctctagacctagctgctgttgaagttccatctacaaatggataagacttccattctgtcttagtgtatacgaatcattgatggagcaatatctttgtatcttgctaaagatattgctccctttttttggccaatcattgtgggtataatggtagatgccctagaccaagtgactattatttctttctcctcccttatgtttagtttttcaatttttgccgataaatgattagctacaaaaggatttttttttattgaacgtgtcacaggggattactcctttattacattacatttttaaaattggcattctatgcccaatatatcgatctaagtatgaaggtaagaataaatacaataatgatgaatggaaaaagaaaaaagctaaaatcttttagctagataaggggcggatgcagccaagtggatcaaggcagtggatttgtgaatccaccatgcgcg

SEQ ID NO: 40 - DNA, 401 bp, Dioscorea cirrhosa
ggtatgcatgaacgtaatgctcacaactttcctttagacttagctgctgttgaagttccatctacaaatggataagacttttgtcttaatgtatatgaatcgttgaaggagcaatatcaatatcttgttcgagcaagaagtttggtattgctccccttttcaatttttcccgataaatgatcaactacaaaaggatttttttttagtgaacgtgtcacagcggattactcctttattttttacatttttaagattggcattctatgttcaatatctcgatctaataaggtaagaataaatttaaatacaataatgatgaatggaaaaagagaaaatcctttagctagggcggatgtagccaagtggatcaaggcagtggatttgtgaatccaccatgcgcg

SEQ ID NO: 41 - DNA, 11 bp, Unknown Sequence
  Description of Unknown Sequence Unknown Aristolochia species sequence
gtactgaaata

SEQ ID NO: 42 - DNA, 14 bp, Unknown Sequence
  Description of Unknown Sequence Unknown Muxiang species sequence
gaactaaaaaagga

SEQ ID NO: 43 - DNA, 134 bp, Unknown Sequence
  Description of Unknown Sequence Unknown Muxiang species sequence
ctaaaatagataaaaattataattttgattatttattgcttttatttcagaaataagaaagaaataatatgctcttttttttatgttaatggaaaaataaaatatagtaatagtagataatactagataatagg

SEQ ID NO: 44 - DNA, 11 bp, Unknown Sequence
  Description of Unknown Sequence Unknown Aristolochia species sequence
atcgactatca

SEQ ID NO: 45 - DNA, 13 bp, Unknown Sequence
  Description of Unknown Sequence Unknown Aristolochia species sequence
tttttcacttact

SEQ ID NO: 46 - DNA, 13 bp, Unknown Sequence
  Description of Unknown Sequence Unknown Fangji species sequence
tttctcgttcact

SEQ ID NO: 47 - DNA, 11 bp, Unknown Sequence
  Description of Unknown Sequence Unknown Aristolochia species sequence
tccatatatgg

SEQ ID NO: 48 - DNA, 13 bp, Unknown Sequence
  Description of Unknown Sequence Unknown Fangji species sequence
gtttcccgacatc

SEQ ID NO: 49 - DNA, 10 bp, Unknown Sequence
  Description of Unknown Sequence Unknown Baiying species sequence
gaatactata

SEQ ID NO: 50 - DNA, 13 bp, Unknown Sequence
  Description of Unknown Sequence Unknown Baiying species sequence
acaagcctttatg

SEQ ID NO: 51 - DNA, 17 bp, Unknown Sequence
  Description of Unknown Sequence Unknown Aristolochia species sequence
tttatttaggtaagtta

SEQ ID NO: 52 - DNA, 17 bp, Unknown Sequence
  Description of Unknown Sequence Unknown Baiying species sequence
tttgagaaaatttttta

SEQ ID NO: 53 - DNA, 11 bp, Unknown Sequence
  Description of Unknown Sequence Unknown Baiying species sequence
gagattggaat

SEQ ID NO: 54 - DNA, 12 bp, Unknown Sequence
  Description of Unknown Sequence Unknown Baiying species sequence
gaaaagaaagga

SEQ ID NO: 55 - DNA, 93 bp, Unknown Sequence
  Description of Unknown Sequence Unknown Baiying species sequence
tgttcctcttgttgctaatgttactatatcttttttatttcatttcacaaaaaatataatttttacttcatattcttatctttgaaataataa

SEQ ID NO: 56 - DNA, 49 bp, Unknown Sequence
  Description of Unknown Sequence Unknown Baiying species sequence
aaataataatatcattgaaataagaaagaagagaaatattcgaacttta

SEQ ID NO: 57 - DNA, 27 bp, Unknown Sequence
  Description of Unknown Sequence Unknown Aristolochia species sequence
tacgtacaaatgcccatccatatatgg

SEQ ID NO: 58 - DNA, 45 bp, Unknown Sequence
  Description of Unknown Sequence Unknown Dabaihe species sequence
tacctttgtatcttgctaaagatattgctccctttttttggccaa

SEQ ID NO: 59 - DNA, 22 bp, Unknown Sequence
  Description of Unknown Sequence Unknown Dabaihe species sequence
aagctaaaatcttttagctaga

SEQ ID NO: 60 - DNA, 18 bp, Unknown Sequence
  Description of Unknown Sequence Unknown Shuliang species sequence
atctttatttgttttgtt

SEQ ID NO: 61 - DNA, 11 bp, Unknown Sequence
  Description of Unknown Sequence Unknown Aristolochia species sequence
tcagaagcaga

SEQ ID NO: 62 - DNA, 14 bp, Unknown Sequence
  Description of Unknown Sequence Unknown Shuliang species sequence
tcaaccgaagttga

SEQ ID NO: 63 - DNA, 18 bp, Unknown Sequence
  Description of Unknown Sequence Unknown Aristolochia species sequence
atcgactatcacaccaga

SEQ ID NO: 64 - DNA, 15 bp, Unknown Sequence
  Description of Unknown Sequence Unknown Shuliang species sequence
atcattgattctaga

SEQ ID NO: 65 - DNA, 18 bp, Unknown Sequence
  Description of Unknown Sequence Unknown Aristolochia species sequence
tgcatcgagaatggtcgg

SEQ ID NO: 66 - DNA, 23 bp, Unknown Sequence
  Description of Unknown Sequence Unknown Shuliang species sequence
tgcgagaaaagaaaatggttggg

SEQ ID NO: 67 - DNA, 11 bp, Unknown Sequence
  Description of Unknown Sequence Unknown Aristolochia species sequence
aatttcagaag

SEQ ID NO: 68 - DNA, 11 bp, Unknown Sequence
  Description of Unknown Sequence Unknown Aristolochia species sequence
actatcacacc

SEQ ID NO: 69 - DNA, 12 bp, Unknown Sequence
  Description of Unknown Sequence Unknown Aristolochia species sequence
ctcaattcacta

SEQ ID NO: 70 - DNA, 20 bp, Artificial Sequence
  Description of Artificial Sequence Synthetic primer
cgaaatcggtagacgctacg

SEQ ID NO: 71 - DNA, 20 bp, Artificial Sequence
  Description of Artificial Sequence Synthetic primer
atttgaactggtgacacgag

SEQ ID NO: 72 - DNA, 22 bp, Artificial Sequence
  Description of Artificial Sequence Synthetic primer
ggtatgcatgaacgtaatgctc

SEQ ID NO: 73 - DNA, 23 bp, Artificial Sequence
  Description of Artificial Sequence Synthetic primer
cgcgcatggtggattcacaaatc